SEQUENCE LISTING (108 sequences)

SEQ ID NO: 1 - RNA, 21 nt, Homo sapiens
ugucaguuugucaaauacccc

SEQ ID NO: 2 - RNA, 21 nt, Homo sapiens
uagguaguuucauguuguugg

SEQ ID NO: 3 - RNA, 18 nt, Homo sapiens
ucuacagugcacgugucu

SEQ ID NO: 4 - RNA, 22 nt, Homo sapiens
cauugcacuugucucggucuga

SEQ ID NO: 5 - RNA, 23 nt, Homo sapiens
acuccauuuguuuugaugaugga

SEQ ID NO: 6 - RNA, 23 nt, Homo sapiens
auucuaauuucuccacgucuuug

SEQ ID NO: 7 - RNA, 22 nt, Homo sapiens
uuuguucguucggcucgcguga

SEQ ID NO: 8 - RNA, 23 nt, Homo sapiens
ugucugcccgcaugccugccucu

SEQ ID NO: 9 - RNA, 21 nt, Homo sapiens
agacccuggucugcacucuau

SEQ ID NO: 10 - RNA, 21 nt, Homo sapiens
gugucuuuugcucugcaguca

SEQ ID NO: 11 - RNA, 20 nt, Homo sapiens
aggaauguuccuucuuugcc

SEQ ID NO: 12 - RNA, 21 nt, Homo sapiens
ugaaggucuacugugugccag

SEQ ID NO: 13 - RNA, 21 nt, Homo sapiens
ugugacugguugaccagaggg

SEQ ID NO: 14 - RNA, 22 nt, Homo sapiens
ugguugaccauagaacaugcgc

SEQ ID NO: 15 - RNA, 21 nt, Homo sapiens
aucacauugccagggauuucc

SEQ ID NO: 16 - RNA, 23 nt, Homo sapiens
agcuacauugucugcuggguuuc

SEQ ID NO: 17 - RNA, 22 nt, Homo sapiens
aaggagcucacagucuauugag

SEQ ID NO: 18 - RNA, 22 nt, Homo sapiens
gaaguuguucgugguggauucg

SEQ ID NO: 19 - RNA, 22 nt, Homo sapiens
agacuucccauuugaagguggc

SEQ ID NO: 20 - RNA, 22 nt, Homo sapiens
cuggacuuggagucagaaggcc

SEQ ID NO: 21 - RNA, 22 nt, Homo sapiens
aaagugccuccuuuuagagugu

SEQ ID NO: 22 - RNA, 23 nt, Homo sapiens
aaagugcauccuuuuagagguuu

SEQ ID NO: 23 - RNA, 23 nt, Homo sapiens
aucgugcaucccuuuagaguguu

SEQ ID NO: 24 - RNA, 23 nt, Homo sapiens
uaagugcuuccauguuuugguga

SEQ ID NO: 25 - RNA, 23 nt, Homo sapiens
uaagugcuuccauguuugagugu

SEQ ID NO: 26 - RNA, 21 nt, Homo sapiens
gugccgccaucuuuugagugu

SEQ ID NO: 27 - RNA, 22 nt, Homo sapiens
ucucccaacccuuguaccagug

SEQ ID NO: 28 - RNA, 23 nt, Homo sapiens
ugugcaaauccaugcaaaacuga

SEQ ID NO: 29 - RNA, 21 nt, Homo sapiens
aggguaagcugaaccucugau

SEQ ID NO: 30 - RNA, 21 nt, Homo sapiens
ucguaccgugaguaauaaugc

SEQ ID NO: 31 - RNA, 21 nt, Homo sapiens
uucacaguggcuaaguuccgc

SEQ ID NO: 32 - RNA, 22 nt, Homo sapiens
uaacacugucugguaacgaugu

SEQ ID NO: 33 - RNA, 24 nt, Homo sapiens
aaaagugcuuacagugcagguagc

SEQ ID NO: 34 - RNA, 22 nt, Homo sapiens
aagugcugucauagcugagguc

SEQ ID NO: 35 - RNA, 21 nt, Homo sapiens
uggaauguaaagaaguaugua

SEQ ID NO: 36 - RNA, 23 nt, Homo sapiens
ucaagagcaauaacgaaaaaugu

SEQ ID NO: 37 - RNA, 21 nt, Homo sapiens
uugugcuugaucuaaccaugu

SEQ ID NO: 38 - RNA, 21 nt, Homo sapiens
uaaagcuagauaaccgaaagu

SEQ ID NO: 39 - RNA, 22 nt, Homo sapiens
uggaauguaaggaagugugugg

SEQ ID NO: 40 - RNA, 22 nt, Drosophila melanogaster
uggaauguaaagaaguauggag

SEQ ID NO: 41 - DNA, 20 bp, Homo sapiens
  misc_feature 11, 17: n is any nucleic acid
tggaatgtaangaagtntgt

SEQ ID NO: 42 - RNA, 71 nt, Homo sapiens
ugggaaacauacuucuuuauaugcccauauggaccugcuaagcuauggaauguaaagaaguauguaucuca

SEQ ID NO: 43 - RNA, 85 nt, Homo sapiens
accuacucagaguacauacuucuuuauguacccauaugaacauacaaugcuauggaauguaaagaaguauguauuuuugguaggc

SEQ ID NO: 44 - RNA, 77 nt, Mus musculus
gcuugggacacauacuucuuuauaugcccauaugaaccugcuaagcuauggaauguaaagaaguauguauuucaggc

SEQ ID NO: 45 - RNA, 72 nt, Mus musculus
ucagagcacauacuucuuuauguacccauaugaacauucagugcuauggaauguaaagaaguauguauuuug

SEQ ID NO: 46 - RNA, 86 nt, Homo sapiens
ugcuucccgaggccacaugcuucuuuauauccccauauggauuacuuugcuauggaauguaaggaagugugugguuucggcaagug

SEQ ID NO: 47 - RNA, 73 nt, Mus musculus
ccaggccacaugcuucuuuauauccucauagauaucucagcacuauggaauguaaggaagugugugguuuugg

SEQ ID NO: 48 - RNA, 91 nt, Drosophila melanogaster
uucagccuuugagaguuccaugcuuccuugcauucaauaguuauauucaagcauauggaauguaaagaaguauggagcgaaaucuggcgag

SEQ ID NO: 49 - DNA, 65 bp, Homo sapiens
  misc_feature (2)..(4): n is a or g or c or t
gnnnacatncttctttatntncccatannnanntncnnnctatggaatgtaagaagtntgtnnnt

SEQ ID NO: 50 - RNA, 22 nt, Homo sapiens
uauugcacuugucccggccugu

SEQ ID NO: 51 - RNA, 21 nt, Mus musculus
uauugcacuugucccggccug

SEQ ID NO: 52 - RNA, 22 nt, Drosophila melanogaster
cauugcacuugucccggccuau

SEQ ID NO: 53 - RNA, 22 nt, Homo sapiens
uauugcacucgucccggccucc

SEQ ID NO: 54 - RNA, 22 nt, Drosophila melanogaster
aauugcacuagucccggccugc

SEQ ID NO: 55 - RNA, 22 nt, Drosophila melanogaster
uauugcacacuucccggccuuu

SEQ ID NO: 56 - RNA, 22 nt, Drosophila melanogaster
uauugcacauucaccggccuga

SEQ ID NO: 57 - RNA, 22 nt, Drosophila melanogaster
uauugcacuugagacggccuga

SEQ ID NO: 58 - RNA, 22 nt, Drosophila melanogaster
uauugcacuuuucacagcccga

SEQ ID NO: 59 - DNA, 19 bp, Homo sapiens
  misc_feature (9): n is a or g or c or t
attgcactnntcncggcct

SEQ ID NO: 60 - RNA, 84 nt, Homo sapiens
ggccaguguugagaggcggagacuugggcaauugcuggacgcugcccugggcauugcacuugucucggucugacagugccggcc

SEQ ID NO: 61 - RNA, 78 nt, Homo sapiens
cuuucuacacagguugggaucgguugcaaugcuguguuucuguaugguauugcacuugucccggccuguugaguuugg

SEQ ID NO: 62 - RNA, 80 nt, Mus musculus
cuuucuacacagguugggauuugucgcaaugcuguguuucucuguaugguauugcacuugucccggccuguugaguuugg

SEQ ID NO: 63 - RNA, 75 nt, Homo sapiens
ucaucccuggguggggauuuguugcauuacuuguguucuauauaaaguauugcacuugucccggccuguggaaga

SEQ ID NO: 64 - RNA, 91 nt, Mus musculus
ugcccauucauccacagguggggauugguggcauuacuuguguuagauauaaaguauugcacuugucccggccugaggaagaaagaggguu

SEQ ID NO: 65 - RNA, 95 nt, Drosophila melanogaster
aauaugaauuucccguaggacgggaaggugucaacguuuugcauuucgaauaaacauugcacuugucccggccuaugggcgguuuguaauaaaca

SEQ ID NO: 66 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 67 - RNA, 83 nt, Mus musculus
ggugggcgggagggacgggacguggugcaguguuguucuuuccccugccaauauugcacucgucccggccuccggcccccucg

SEQ ID NO: 68 - RNA, 100 nt, Drosophila melanogaster
uaaaacgucaccugauguaggccgugcccagugcuuauuuguugcauuuucgaaauacaaauugcacuagucccggccugcaaugagugucgcagucgac

SEQ ID NO: 69 - RNA, 89 nt, Drosophila melanogaster
aacauaaacauuugcagggcgggucgugugucaguguauuuauaucuuagcuauauugcacacuucccggccuuuaaauguccaauguu

SEQ ID NO: 70 - RNA, 84 nt, Drosophila melanogaster
ucuagaucauuuuucggacgguauauggguuaauauuucauuugucgaauauauugcacauucaccggccugaaaauaucaaga

SEQ ID NO: 71 - RNA, 66 nt, Drosophila melanogaster
gauuugguucgucacaagggcaauucugcauuuuuuaacuaguauugcacuugagacggccugauu

SEQ ID NO: 72 - RNA, 71 nt, Drosophila melanogaster
auuuucugcugcggaugggggcaguacuguuuuuuuaacauugaguauugcacuuuucacagcccgaaaau

SEQ ID NO: 73 - DNA, 20 bp, Homo sapiens
  misc_feature (9): n is a or g or c or t
attgcactnngtcncggcct

SEQ ID NO: 74 - RNA, 21 nt, Homo sapiens
gagugccuucuuuuggagcgu

SEQ ID NO: 75 - RNA, 18 nt, Homo sapiens
ugcuuccuuucagagggu

SEQ ID NO: 76 - RNA, 23 nt, Homo sapiens
aucgugcaucccuuuagaguguu

SEQ ID NO: 77 - RNA, 22 nt, Homo sapiens
ucgugcaucccuuuagaguguu

SEQ ID NO: 78 - RNA, 22 nt, Homo sapiens
aucgugcauccuuuuagagugu

SEQ ID NO: 79 - RNA, 21 nt, Homo sapiens
aaagcgcuucccuuugcugga

SEQ ID NO: 80 - RNA, 22 nt, Homo sapiens
caaagcgcuccccuuuagaggu

SEQ ID NO: 81 - RNA, 22 nt, Homo sapiens
caaagcgcuucucuuuagagug

SEQ ID NO: 82 - RNA, 21 nt, Homo sapiens
caaagcgcuucccuuuggagc

SEQ ID NO: 83 - RNA, 22 nt, Homo sapiens
aaagcgcuucccuucagagugu

SEQ ID NO: 84 - RNA, 21 nt, Homo sapiens
aaagcgcuucucuuuagagga

SEQ ID NO: 85 - RNA, 25 nt, Homo sapiens
aaagugcauccuuuuagaguguuac

SEQ ID NO: 86 - RNA, 23 nt, Homo sapiens
aaagugcauccuuuuagagguuu

SEQ ID NO: 87 - RNA, 22 nt, Homo sapiens
aaagugcaucuuuuuagaggau

SEQ ID NO: 88 - RNA, 23 nt, Homo sapiens
caaagugccucccuuuagagugu

SEQ ID NO: 89 - RNA, 22 nt, Homo sapiens
aaagugccuccuuuuagagugu

SEQ ID NO: 90 - RNA, 22 nt, Homo sapiens
aaagugcuucccuuuggacugu

SEQ ID NO: 91 - RNA, 21 nt, Homo sapiens
aaagugcuuccuuuuagaggg

SEQ ID NO: 92 - RNA, 21 nt, Homo sapiens
aaagugcuuccuuuuugaggg

SEQ ID NO: 93 - RNA, 22 nt, Homo sapiens
aagugcuuccuuuuagaggguu

SEQ ID NO: 94 - RNA, 23 nt, Homo sapiens
aaagugcuuccuuuuagaggguu

SEQ ID NO: 95 - RNA, 23 nt, Homo sapiens
aaagugcuucucuuugguggguu

SEQ ID NO: 96 - RNA, 22 nt, Homo sapiens
aacgcacuucccuuuagagugu

SEQ ID NO: 97 - RNA, 23 nt, Homo sapiens
aaaaugguucccuuuagaguguu

SEQ ID NO: 98 - RNA, 21 nt, Homo sapiens
aacgcgcuucccuauagaggg

SEQ ID NO: 99 - RNA, 21 nt, Homo sapiens
gaaggcgcuucccuuuggagu

SEQ ID NO: 100 - RNA, 21 nt, Homo sapiens
cuccagagggaugcacuuucu

SEQ ID NO: 101 - RNA, 21 nt, Homo sapiens
cucuagagggaagcacuuucu

SEQ ID NO: 102 - RNA, 21 nt, Homo sapiens
cugcaaagggaagcccuuucu

SEQ ID NO: 103 - RNA, 22 nt, Homo sapiens
  misc_feature (7): n is a or g or c or u
aagugcnuccnnnuuunnngng

SEQ ID NO: 104 - RNA, 19 nt, Homo sapiens
  misc_feature (5): n is a or g or c or u
gugcnuccnnnuuunnngn

SEQ ID NO: 105 - DNA, 20 bp, Artificial Sequence
  Synthetic oligonucleotide
atggcaaccaagaaagcaag

SEQ ID NO: 106 - DNA, 23 bp, Artificial Sequence
  Synthetic oligonucleotide
ggtccacagtagtttttcgtaag

SEQ ID NO: 107 - DNA, 18 bp, Artificial Sequence
  Synthetic oligonucleotide
tgaaggtcggagtcaacg

SEQ ID NO: 108 - DNA, 18 bp, Artificial Sequence
  Synthetic oligonucleotide
gggtcattgatggcaaca

(19) United States
(12) Patent Application Publication
(10) Pub. No.: US 2015/0267193 A1
(43) Pub. Date: Sep. 24, 2015

(54) MicroRNAs and Methods of Using Same

(71) Applicant: Ramanuj Dasgupta, New York, NY (US)

(72) Inventor: Ramanuj Dasgupta, New York, NY (US)

(21) Appl. No.: 13/651,672

(22) Filed: Oct. 15, 2012

Related U.S. Application Data
(60) Provisional application No. 61/627,628, filed on Oct. 14, 2011.

(51) Int. Cl. C12N 15/113; C12Q 1/68

(52) U.S. Cl. CPC C12N 15/113; C12Q 1/6886; C12N 2310/141; C12Q 2600/112; C12Q 2600/178

(57) ABSTRACT

MicroRNAs (miRs) and methods and uses thereof for modulating Wnt signaling pathways are described herein. More particularly, miRs and compositions thereof and methods for treating a cancer associated with an elevated or hyper-activated Wnt signaling pathway using same, as well as miRs for use in treating cancers associated with an elevated or hyper-activated Wnt signaling pathway and for use in the preparation of a medicament for treating a cancer associated with an elevated or hyper-activated Wnt signaling pathway in a subject are described herein

### CROSS REFERENCE TO RELATED APPLICATIONS

This application claims priority under 35 USC §119(e) from U.S. Provisional Application Ser. No. 61/627,628, filed Oct. 14, 2011, which application is herein specifically incorporated by reference in its entirety.

### GOVERNMENTAL SUPPORT

The research leading to the present invention was supported, at least in part, by a Department of Defense (DOD)/BCRP concept award W81XWH-07-1-0541 and DOD-BC093088. Accordingly, the Government has certain rights in the invention.

### FIELD OF INVENTION

The compositions and methods described herein relate to microRNAs (miRs) and their role in various cancers. More particularly, compositions and methods described herein relate to miRs and their role in cancers influenced by the Wnt pathway and modulation thereof.

### BACKGROUND OF INVENTION

The citation of references herein shall not be construed as an admission that such is prior art to the present invention.

Wnts/wingless (wg) are a family of conserved signaling molecules that have been shown to regulate a plethora of fundamental developmental and cell biological processes, including cell proliferation, differentiation and cell polarity [Miller et al. Oncogene 18, 7860-72 (1999); Polakis. Genes Dev 14, 1837-51 (2000); Wodarz et al. Annu Rev Cell Dev Biol 14, 59-88 (1998)]. Mutations in the Wnt genes or in those genes encoding regulators of the Wnt/wg signaling pathway can cause devastating birth defects, including debilitating abnormalities of the central nervous system, axial skeleton, limbs, and occasionally other organs [Ciruna et al. Nature 439, 220-4 (2006); Grove et al. Development 125, 2315-25 (1998); Jiang et al. Dev Dyn 235, 1152-66 (2006); Kokubu et al. Development 131, 5469-80 (2004); Miyoshi et al. Breast Cancer Res 5, 63-8 (2003); Shu et al. Development 129, 4831-42 (2002); Staal et al. Hematol J 1, 3-6 (2000)]. Aberrant Wnt signaling has also been linked to human disease, such as hepatic, colorectal, breast and skin cancers [Miyoshi et al. supra (2003); Miyoshi et al. Oncogene 21, 5548-56 (2002); Moon et al. Nat Rev Genet 5, 691-701 (2004)]. Activating mutations of beta-catenin have also been found in around 5% of prostate cancers [Chesire et al., The Prostate 45, 323 (2000); Voeller et al., Cancer research 58, 2520 (1998)]. Mutation of APC has been found in 14% in one study [Gerstein et al., Genes, chromosomes & cancer 34, 9 (2002)] and 3% in another [Watanabe et al., Japanese journal of clinical oncology 26, 77 (1996)]. Over 20% of advanced prostate cancer, 77% of prostatic lymph node metastases and 85% of prostatic skeletal metastases have been reported to exhibit increased nuclear beta-catenin, as shown by immunohistochemistry [Chen et al., Cancer 101, 1345 (2004)]. The ligands of Wnt-pathway, Wnt1, Wnt2 and Wnt5a are, moreover, up-regulated in prostate cancer samples [Chen et al., Cancer 101, 1345 (2004); Katoh, International journal of oncology 19, 1003 (2001); Usui et al., Nihon Sanka Fujinka Gakkai zasshi 44, 703 (1992)]. Immunohistochemistry has revealed that one inhibitor of the Wnt-pathway, WIF1, was down-regulated in prostate cancer [Wissmann et al., The Journal of pathology 201, 204 (2003)].

Colon and gastrointestinal cancers are amongst the leading causes of cancer-related mortality and they all have been linked, together with many other cancers, to mutations in components of the Wnt/β-catenin pathway [1]. Therefore there is a major interest in targeting the activity of this pathway using genetic and chemical therapeutic tools. The promise of one emerging approach rests upon the therapeutic potential of small interfering RNAs (siRNAs) and microRNAs (miRs). miRs are small RNAs (˜ca. 22 nt in length) that regulate the level of mRNAs and proteins by targeted degradation of specific mRNAs and/or repression of their translation [2], [3]. Functions of miRs have been identified in apoptosis, proliferation, differentiation [2] and stem cell maintenance [4]. They have also been associated with cancer progression and metastasis [5], [6], [7]. Steady-state expression profiles of certain miRs have often been found to be deregulated in cancers and can aid in prognosis [8], [9], [10]. Individual miRs that have been reported to down-regulate oncogenes such as ras [11] are called anti-oncomiRs and inhibit cancer proliferation. Others, termed oncomiRs, function in a cancer-supportive or inductive manner by down-regulating tumor-suppressors such as p53 [12], [13] and inducing proliferation and/or metastasis. The Wnt/β-catenin pathway is often found to be elevated in gastrointestinal, breast and colon cancers among others and there is strong evidence for a role of hyper-activated Wnt signaling in cancer initiation and progression[14], [15], [16], [17], [18]. The key element of Wnt signaling is the transcriptional co-activator role of β-catenin, whose level is tightly controlled by a destruction complex including a scaffold protein, Axin-1, APC, and GSK-3β, a kinase that phosphorylates β-catenin, which results in its ubiquitination and subsequent proteasomal degradation[18], [19]. Wnt signaling via LRP5/6/Frizzled receptors and cytosolic Dsh among other factors, destabilizes this destruction complex, which leads to accumulation of β-catenin and its association with TCF/LEF family transcription factors in the nucleus to activate specific target genes [18], [19]. Negative regulators of Wnt signaling like APC and Axin function as tumor-suppressors and the viability of some cancer cell lines is believed to be Wnt-dependent [20], [21], [22], [23], [24], [25].

Several publications and patent documents are referenced in this application in order to more fully describe the state of the art to which this invention pertains. The disclosure of each of these publications and documents is incorporated by reference herein.

### SUMMARY OF THE INVENTION

MicroRNAs (miRs) and the Wnt pathway are known to be dysregulated in human cancers and play key roles during cancer initiation and progression. To identify miRs that can modulate the activity of the Wnt pathway we performed a cell-based overexpression screen of 470 miRs in human HEK293 cells. We identified 38 candidate miRs that either activate or repress the Wnt pathway. A literature survey of all verified candidate miRs revealed that the Wnt-repressing miRs tend to be anti-oncomiRs and down-regulated in cancers while Wnt-activating miRs tend to be oncomiRs and upregulated during tumorigenesis. Epistasis-based functional validation of three candidate miRs, miR-1, miR-25 and miR-613, confirmed their inhibitory role in repressing the Wnt pathway and suggest that while miR-25 may function at the level of β-catenin (β-cat), miR-1 and miR-613 act upstream of β-cat. Both miR-25 and miR-1 inhibit cell proliferation and viability during selection of human colon cancer cell lines that exhibit dysregulated Wnt signaling. Finally, transduction of miR-1 expressing lentiviruses into primary mammary organoids derived from Conductin-lacZ mice significantly reduced the expression of the Wnt-sensitive β-gal reporter. In summary, these findings suggest the potential use of Wnt-modulating miRs as diagnostic and therapeutic tools in Wnt-dependent diseases, such as cancer.

In accordance with these findings, a method for modulating Wnt signaling pathways in a cell is presented, the method comprising contacting the cell with at least one microRNA listed in FIG. 1 or FIG. 2 or expressing at least one microRNA listed in FIG. 1 or 2 in the cell, wherein the at least one microRNA modulates expression of at least one nucleic acid sequence encoding a component of the Wnt pathway in the cell. The nucleic acid sequences of microRNA listed in FIG. 2 are designated SEQ ID NOs: 1-38, respectively (counting from the top to the bottom of the list). In a particular aspect thereof, SEQ ID NOs: 1-30 and 33-38 are envisioned in this method. In a more particular aspect thereof, microRNA are grouped according to structure/function as inhibitors of Wnt signaling pathways or activators of Wnt signaling pathways. Exemplary inhibitors of Wnt signaling pathways comprise microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (also referred to herein as microRNA-1; SEQ ID NO: 35), 335 (SEQ ID NO: 36), 218-1 (SEQ ID NO: 37), and 9 (SEQ ID NO: 38). Exemplary activators of Wnt signaling pathways comprise microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), and 512-2-3p (SEQ ID NO: 34).

In a particular embodiment thereof, the cell is a cancer cell. In a more particular embodiment, the Wnt signaling pathway is elevated or hyper-activated in the cancer cell and the at least one microRNA reduces or inhibits Wnt signaling pathways. In a further embodiment the at least one microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38). In another embodiment, the at least one microRNA comprises a consensus sequence as set forth in FIG. 7 or 9. In a further embodiment, the at least one microRNA comprises a consensus sequence as set forth in FIG. 7 or 9 and is a member of either of the miR-1/206 family or the miR-25/92 family. In a particular embodiment, the consensus sequence is a GGAAUGU seed sequence. In a particular embodiment thereof, the at least one microRNA is miR-1 or miR-613. In another particular embodiment, the consensus sequence comprises a UUGCAC seed sequence and the microRNA is miR-25, miR-92a, or miR-92b. In an even more particular embodiment, the at least one microRNA is miR-1, miR-25 or miR-613.

In a further embodiment, the method for modulating Wnt signaling pathways in a cell is performed by contacting with or expressing at least one microRNA that activates the Wnt signaling pathway. Applications for which increasing or activating the Wnt signaling pathway is desirable include the treatment of bone degenerative disorders wherein bones become progressively more brittle. Osteoporosis is an exemplary bone degenerative disorder treatable using the Wnt pathway activating microRNA described herein. In a further embodiment thereof, the at least one microRNA is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34). In a more particular embodiment, the at least one microRNA is microRNA 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In another embodiment, the at least one microRNA comprises a consensus sequence as set forth in FIG. 13. In a more particular embodiment, the at least one microRNA is a member of either of the miR-302 family or the miR-515 family.

In a further aspect, a method for treating a subject with a cancer associated with an elevated or hyper-activated Wnt signaling pathway is presented, the method comprising administering to the subject at least one inhibitory microRNA shown in either of FIG. 1 or FIG. 2, wherein the at least one inhibitory microRNA reduces or inhibits the elevated or hyper-activated Wnt signaling pathway.

In a still further aspect, a method for reducing or inhibiting Wnt signaling pathways in a subject with a cancer associated with an elevated or hyper-activated Wnt signaling pathway is presented, the method comprising administering to the subject at least one inhibitory microRNA shown in either of FIG. 1 or FIG. 2, wherein the at least one inhibitory microRNA reduces or inhibits the Wnt signaling pathways in the subject.

In an embodiment of these methods, the at least one inhibitory microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38). In another embodiment, the at least one microRNA comprises a consensus sequence as set forth in FIG. 7 or 9. In a further embodiment, the at least one microRNA comprises a consensus sequence as set forth in FIG. 7 or 9 and is a member of either of the miR-1/206 family or the miR-25/92 family. In a particular embodiment, the consensus sequence is a GGAAUGU seed sequence. In a more particular embodiment thereof, the at least one microRNA is miR-1 or miR-613. In another particular embodiment, the consensus sequence comprises a UUGCAC seed sequence and the microRNA is miR-25, miR-92a, or miR-92b. In an even more particular embodiment, the at least one microRNA is miR-1, miR-25 or miR-613.

In a further aspect, the at least one inhibitory microRNA utilized in the methods described herein is modified to improve therapeutic efficacy as described herein and known in the art. In a particular embodiment thereof, the 3′ or 5′ end is modified to improve therapeutic efficacy. Delivery of the at least one microRNA may be achieved using a variety of means, including liposomes, lipidoids, nanovesicles and the like as described herein and known in the art.

MicroRNA described herein, whether inhibitory microRNA or activating microRNA with respect to their effect on Wnt signaling pathways, can be administered by a variety of routes, including without limitation, intravenously, intraperitoneally, orally, and/or in a localized fashion so as to delivery the microRNA in a region of the body wherein it is desirable to modulate the activity of Wnt signaling pathways. With regard to a method for reducing or inhibiting Wnt signaling pathways in a subject with a cancer associated with an elevated or hyper-activated Wnt signaling pathway or for treating a cancer associated with an elevated or hyper-activated Wnt signaling pathway, it may be desirable to deliver an inhibitory microRNA intratumorally or in a localized fashion in the immediate area of the tumor.

Also encompassed herein is a method for diagnosing a cancer associated with an elevated or hyper-activated Wnt signaling pathway in a subject, the method comprising determining the level of at least one microRNA shown in either of FIG. 1 or FIG. 2, wherein the at least one microRNA activates the Wnt signaling pathway, and wherein detection of upregulated levels of the at least one microRNA in the subject is diagnostic for the presence of the cancer in the subject. In an embodiment thereof, the at least one microRNA is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34). In a more particular embodiment, the at least one microRNA is microRNA 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In another embodiment, the at least one microRNA comprises a consensus sequence as set forth in FIG. 13. In a more particular embodiment, the at least one microRNA is a member of either of the miR-302 family or the miR-515 family.

Also encompassed herein is a method for determining the efficacy of a therapeutic agent for treating a cancer associated with an elevated or hyper-activated Wnt signaling pathway in a subject, the method comprising determining the level of at least one microRNA shown in either of FIG. 1 or FIG. 2, wherein the at least one microRNA activates the Wnt signaling pathway, and wherein detection of reduced levels of the at least one microRNA in the subject following administration of the therapeutic agent as compared to prior to administration of the therapeutic agent indicates that the therapeutic agent is efficacious for treating the cancer in the subject. In an embodiment thereof, the at least one microRNA is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34). In a more particular embodiment, the at least one microRNA is microRNA 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In another embodiment, the at least one microRNA comprises a consensus sequence as set forth in FIG. 13. In a more particular embodiment, the at least one microRNA is a member of either of the miR-302 family or the miR-515 family.

Also encompassed herein is a method for determining the efficacy of a therapeutic agent for treating a cancer associated with an elevated or hyper-activated Wnt signaling pathway in a subject, the method comprising determining the level of at least one microRNA shown in either of FIG. 1 or FIG. 2, wherein the at least one microRNA inhibits the Wnt signaling pathway, and wherein detection of increased levels of the at least one microRNA in the subject following administration of the therapeutic agent as compared to prior to administration of the therapeutic agent indicates that the therapeutic agent is efficacious for treating the cancer in the subject. In an embodiment thereof, the at least one microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38). In another embodiment, the at least one microRNA comprises a consensus sequence as set forth in FIG. 7 or 9. In a further embodiment, the at least one microRNA comprises a consensus sequence as set forth in FIG. 7 or 9 and is a member of either of the miR-1/206 family or the miR-25/92 family. In a particular embodiment, the consensus sequence is a GGAAUGU seed sequence. In a more particular embodiment thereof, the at least one microRNA is miR-1 or miR-613. In another particular embodiment, the consensus sequence comprises a UUGCAC seed sequence and the microRNA is miR-25, miR-92a, or miR-92b. In an even more particular embodiment, the at least one microRNA is miR-1, miR-25 or miR-613.

Methods described herein may be performed using a sample isolated from the subject. Samples include, without limitation, fresh frozen or fixed tissue (e.g., paraffin-fixed tissue) of tumor biopsies collected during surgery. In a particular embodiment, the subject is a mammal. In a more particular embodiment, the mammal is a human.

Also envisioned herein is a method for screening to identify a modulator of at least one microRNA shown in either of FIG. 1 or FIG. 2, the method comprising contacting the at least one microRNA with an agent to determine if the activity of the at least one microRNA is altered in the presence of the agent relative to the activity in the absence of the agent. In a particular embodiment, the at least one microRNA activates the Wnt signaling pathway and the agent identified reduces the activity of the at least one microRNA. In a further embodiment, the at least one microRNA represses the Wnt signaling pathway and the agent identified increases the activity of the at least one microRNA.

In a further aspect, at least one inhibitory microRNA according to either of FIG. 1 or FIG. 2 for use in treating a cancer associated with an elevated or hyper-activated Wnt signaling pathway in a subject is presented, wherein the at least one inhibitory microRNA is administered to the subject to reduce or inhibit the elevated or hyper-activated Wnt signaling pathway and thereby treat the cancer.

In another aspect, use of at least one inhibitory microRNA according to either of FIG. 1 or FIG. 2 in the preparation of a medicament for treating a cancer associated with an elevated or hyper-activated Wnt signaling pathway in a subject is presented, wherein the medicament reduces or inhibits the elevated or hyper-activated Wnt signaling pathway.

In embodiments thereof, the at least one inhibitory microRNA for use in treating a cancer associated with an elevated or hyper-activated Wnt signaling pathway in a subject or use of the at least one inhibitory microRNA in the preparation of a medicament for treating a cancer associated with an elevated or hyper-activated Wnt signaling pathway in a subject calls for an inhibitory microRNA wherein the microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38). In another embodiment, the at least one inhibitory microRNA comprises a consensus sequence as set forth in FIG. 7 or 9. In a further embodiment, the at least one inhibitory microRNA comprises a consensus sequence as set forth in FIG. 7 or 9 and is a member of either of the miR-1/206 family or the miR-25/92 family. In a particular embodiment, the consensus sequence is a GGAAUGU seed sequence. In a more particular embodiment thereof, the at least one inhibitory microRNA is miR-1 or miR-613. In another particular embodiment, the consensus sequence comprises a UUGCAC seed sequence and the microRNA is miR-25, miR-92a, or miR-92b. In an even more particular embodiment, the at least one inhibitory microRNA is miR-1, miR-25 or miR-613.

In a further aspect, the at least one inhibitory microRNA for use in treating a cancer associated with an elevated or hyper-activated Wnt signaling pathway or for use in the preparation of a medicament for treating a cancer associated with an elevated or hyper-activated Wnt signaling pathway is modified to improve therapeutic efficacy as described herein and known in the art. In a particular embodiment thereof, the 3′ or 5′ end is modified to improve therapeutic efficacy. The at least one inhibitory microRNA may be encapsulated in a liposome, lipidoid, or nanovesicle delivery vehicle. The at least one inhibitory microRNA may be administered via intradermal, intramuscular, intravenous, intraperitoneal, intratumoral, oral, rectal, buccal, or intranasal administration and/or in a localized fashion in the immediate area of the tumor.

In a further aspect, at least one activator/enhancer microRNA according to either of FIG. 1 or FIG. 2 for use in treating a bone degenerative disorder in a subject is presented, wherein the at least one inhibitory microRNA is administered to the subject to increase or enhance Wnt signaling pathways and thereby treat the bone degenerative disorder (e.g., osteoporosis). In another aspect, use of at least one activator/enhancer microRNA according to either of FIG. 1 or FIG. 2 in the preparation of a medicament for treating a bone degenerative disorder (e.g., osteoporosis) in a subject is presented, wherein the medicament increases or enhances Wnt signaling pathways in the affected cells. In an embodiment thereof, the at least one activator/enhancer microRNA is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34). In a more particular embodiment, the at least one microRNA is microRNA 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In another embodiment, the at least one microRNA comprises a consensus sequence as set forth in FIG. 13. In a more particular embodiment, the at least one microRNA is a member of either of the miR-302 family or the miR-515 family.

In a further aspect, the at least one activator/enhancer microRNA for use in treating a bone degenerative disorder or for use in the preparation of a medicament for treating a bone degenerative disorder is modified to improve therapeutic efficacy as described herein and known in the art. In a particular embodiment thereof, the 3′ or 5′ end is modified to improve therapeutic efficacy. The at least one activator/enhancer microRNA may be encapsulated in a liposome, lipidoid, or nanovesicle delivery vehicle. The at least one activator/enhancer microRNA may be administered via intradermal, intramuscular, intravenous, intraperitoneal, intratumoral, oral, rectal, buccal, or intranasal administration and/or in a localized fashion in the immediate area of the osteoporotic bone.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1: Validation of the Pre-miR/Wnt3a/STF-report screen in HEK293 cells. (A) Schematic overview of the screening and validation procedure. (B) Target listing of all validated synthetic cherry-pick Hsa-Pre-miRs and the average of all measurements with SEM (N=2, n=8). Color-code: Hsa-Pre-miRs that significantly repressed (green tab) or activated (red tab) the STF reporter of Wnt-pathway activity. Green disc: anti-oncomiR; red disc: oncomiR. Inset: Representative performance of control siRNAs in one of the assays with LiCl or Wnt3a pathway induction. (C) Simplified sketch of important Wnt/β-catenin pathway nodes/switches and its activation sites by axin1/2 siRNAs or LiCl (D) Correlation between Wnt-pathway repressor (R) and activator (A) miRs being anti-oncogenic (anti-oncomiRs) or oncogenic (oncomiRs). Within each column the fraction of Pre-miRs whose expression is up- or down-regulated in cancer is indicated by color.

FIG. 2: Alignment and tree of all validated Wnt-regulating Pre-miRs to represent and visualize sequence similarities. Numbers indicate Pre-miR designation. I. e. 233 stands for the mature hsa-Pre-miR-223 strand. Green: Pre-miR that was identified and verified as Wnt-inhibitory in the STF reporter assay. Red: Pre-miR that was identified as Wnt-activator/synergizer (A) Alignment of all mature strand selected verified Hsa-Pre-miRs, which nucleic acid sequences are designated SEQ ID NOs: 1-38, respectively, in the order in which they are listed (B) Quartet puzzling tree phylogram determined with tree-puzzle to visualize the degree of sequence similarities between all Pre-miRs based on nucleotide substitutions (C) Representative examples of miRs that share a high degree of sequence similarities. Nucleic acid sequences presented therein correspond to SEQ ID NOs: 24, 25, 21, 22, 23, 26, 9, 10, 4, 3, 37, 35, 36, 16, 15, 31, 30, 13, 14, 5, 6, 27, and 28, respectively, in the order in which they are listed. Note the enrichment of sequence similarity with unidirectional Wnt pathway modulation (see also FIG. 8).

FIG. 3: Molecular and functional characterization of candidate Wnt-regulatory miRs. (A) Epistasis experiments with synthetic human Pre-miR-1, Pre-miR-25 and Pre-miR-613. Influence of miRs on different epistatic pathway stimulations with Wnt3a, LiCl, Axin(1+2)-siRNA or S37A stabilized β-catenin that escapes default ubiquitination and degradation. (B) Semi-quantitative Western blotting to measure relative total β-catenin protein level changes ((3-catenin normalized with α-Tubulin level) in HEK293 cells transfected with 50 nM Pre-miR-1, Pre-miR-25 or Pre-miR-613 at day 3 using different pathway inductions (for 1 day) as indicated. (C) Semi-quantitative Western blotting measurement of relative total β-catenin protein level changes in SW480 colon cancer cell (APC deficiency). (D) STF19x reporter assay normalized by CMV/Renilla to measure endogenous pathway activity in Pri-miR-25 stable cancer cell lines (SW480, HCT116) compared to empty vector control stable cells. (E) Psi-check2 reporter assay system to measure the influence of β-catenin CDS mRNA fragment (inserted into the 3′UTR of a Renilla gene) on transcript translation in the presence of indicated miRs. Left: Blue columns represent the empty vector control. Red columns indicate the psi-check2-CTNNB1-CDS vector. Right: normalized psi-check2-CTNNB2 vector values are shown. (F) Viability of transfected cancer cells during G418 selection. Relative amount of cells obtained after the selection procedure to establish HCT116, SW480, and HT29 colorectal cancer cell lines expressing Pri-miR-25-pcDNA3.1(−)-Neomycin compared to empty vector control cells. Asterisk: unpaired student's t-test, (P<0.05).

FIG. 4: Characterization of miR-1 overexpression in HT29 and HEK293 cells. (A) HT29 colon cancer cells expressing pLV-Hsa-Pre-miR-1 or control vectors at day 4 of puromycin selection. (B) HT29 cells expressing pLV-Hsa-Pre-miR-1 or control at day 7 of puromycin selection. (C) Quantification of HT29 cells at day 4 and 7. RFP (red) fluorescence indicates the expression of RFP harboring the intronic miR-fragment. (D) HEK293 cells expressing miR-1 or control at day 3 (left) and day 7 (right) post puromycin selection. RED RFP fluorescence indicates expressing HEK293 cells. (E) LiCl induced Wnt pathway activity in stable miR-lexpressing HEK293 cells.

FIG. 5: Lentiviral expression of miR-1 inhibits expression of the Wnt reporter, axin2/Conductin-LacZ in primary mouse mammary epithelial organoid cultures. (A-A″″) Representative image of organoid transduced with pLV-dsRed (Control) vector. (B-B″″) Representative image of organoid transduced with pLV-miR-1-dsRed lentiviral vector. The organoids transduced with control lentiviral vector (A″) shows significantly higher expression of Axin2-β-gal compared to organoids expressing miR-1 (B″). The dotted lines in panel A represent non-cellular auto-fluorescence, which was excluded from analysis of fluorescence intensity measured by NIS Elements Software. (C) Quantitative measurement of mean levels of β-gal (fluorescence intensity) in six individual organoids transduced with pLV-dsRed (Control) and seven individual organoids transduced with pLV-miRl-dsRed lentiviral vector. The average intensities were calculated for Region of interest (ROI) selected for each organoid using dsRed expression (that represents lentiviral infection) using the NIS Elements Software. Error bars denote standard deviation among samples.

FIG. 6: Results of the primary Wnt/miR screen. (A) Summarized target listing of cherrypick miRs identified as Wnt-modulating in a primary STF19x-based reporter screen in HEK293 cells. (B) Representative section of the primary screen showing the average of quadruplets as normalized values (STF19x TCF-sites firefly divided by CMV-driven Renilla internal control). Identified target miRs are indicated. (C) Graph showing sorted Z-score values of logarithmized screen data for better comparability. (D) Z-Score of log-transformed screen data (LOG-Z) (green), linear regression on values of unchanged values (red), its subtraction form LOG-Z, and division by 0.56 for a better visual representation (blue). Estimation of about 160 (34%) Wnt-modulating miRs with noise exceeding z-score average values, 80 activators and 80 repressors, respectively. Please note: Anticipating a validation rate of 63.3% (see main text) would yield 101 miRs in the library that modulate the Wnt pathway, representing 21.5%.

FIG. 7: Alignment and phylogenetic quartet puzzling trees of investigated miR families (A) Mature and stem-loop miR strand alignment and tree of members of the miR-1/206 family. (B) Mature and stem-loop alignment of members of the miR-25/92 family.

FIG. 8: Alignment and phylogenetic quartet puzzling tree of all validated Wnt3a-modulating stem-loop miRs identified. (A) Phylogenetic tree of the alignment. Light green: Inhibitor Stem-Loop (SL)-miR similarity group. Light-red: activating SL-miR similarity group. Inset: Quantification of SL- and mature miRs that show the same effect on the Wnt pathway. (B) Alignment of all validated Wnt-modulating SL-miR sequences using ClustalW. (C) Comparison and visualization of the similarity groups identified for stem-loop and mature miRs and their effect on the Wnt pathway.

FIG. 9: Screening results and alignment of studied miRs (miR-1/206 and miR-25/92 family) and miRs with a similar seed sequence. (A) STF19x/CMV-RL reporter values measured in the primary screen for miR-1/206 related miRs inheriting the GGAAUGU seed sequence and their alignment. (B) STF19x/CMV-RL reporter values measured in the primary screen for miR-25/92 related miRs inheriting the UUGCAC seed sequence and their alignment. Note that a few nucleotide substitutions could affect modulation of the Wnt-pathway as measured within the primary screen.

FIG. 10: Possible miR-25 binding sites in β-catenin CDS (543-1874) predicted with RNAhybrid and without seed sequence constraints to include non-canonical seed identification. Mfe: minimum free energy is indicated.

FIG. 11: Table with all data mining references for the correlation studies of identified human Wnt-regulatory miRs and their oncogenicity. (T1.R) Oncogenicity of validated miR/Wnt-repressors. (T1.A) Oncogenicity of validated miR/Wnt-synergizer miRs. (T2.R) Expressional changes of validated miR/Wnt-repressors in cancer. (T2.A) Expression changes of validated miR/Wnt-synergizers in cancer.

FIG. 12: Q-PCR result for β-catenin mRNA levels in Hek293 cells transfected with 50 nM of indicated synthetic Pre-miRs or control siRNAs in the presence of 20 mM LiCl. Note: No significant changes of β-catenin mRNA levels could be measured for all miRs tested.

FIG. 13: Extraction of a consensus sequence of identified miRs within the miR-515 and miR-302 family. Alignment to identify a functional consensus in the miR-515 family and its overlap with modulators the miR-302 family in regarding their ability to modulate the Wnt pathway. (A) Alignment of all tested miR-515 family members in the primary Wnt/miR-screen. (B) Tree of related miR-sequences with normalized Wnt/miR-screen values and standard deviations indicated (local plate average corrected). (C) Alignment of all isolated Wnt-synergizing miR-515 family members to identify a functional RNA consensus sequence that could be essential for the activation-potential on the Wnt pathway. Identification of a common consensus sequence between the regulatory miR-515 and miR-302 family members (below).

### DETAILED DESCRIPTION OF THE INVENTION

miRNAs are a class of endogenous, small non-protein coding RNA molecules that regulate gene expression on a post-transcriptional level. miRNAs play a role in a variety of biological functions, including cellular proliferation, differentiation and apoptosis. The mechanism of miRNA mediated regulation of gene targets largely depends on the degree of complementarity between the miRNA and its target or targets. Typically, miRNAs that bind to mRNA targets with imperfect complementarity bring about translational repression of gene targets, while miRNAs that bind to their mRNA targets with perfect complementarity induce target mRNA cleavage.

Emerging evidence suggests that miRNAs play important roles in the carcinogenesis of various human cancers. Indeed, some miRNAs may be involved in cancers as oncogenes and/or tumor suppressors, whereas others are implicated in tumor invasion and metastasis. See, for example, Xia (J Cancer Molecules 4:79-89, 2008) and Budhu et al. (J Hematology & Oncology 3:37, 2010), the entire contents of each of which is incorporated herein in its entirety.

Further to the above, it has been recently suggested that the delivery and use of anti-oncomiRs or inhibiting oncomiR functionality with antagomiRs [26] may serve as a promising therapeutic approach [27]. We therefore hypothesized that identifying and characterizing miRs that specifically modulate the Wnt pathway could provide a basis for the development of novel Wnt-based therapeutics in Wnt-associated diseases, such as cancer. Research in the past few years have implicated some miRs in the regulation of Wnt signaling [28], [29], [30], [31], [32], [33], [34]. Here we report a systematic screening of a library of 470 human synthetic Pre-miRs and identification of 38 miRs that modulate the activity of the Wnt pathway in human HEK293 cells. Secondary validation and functional testing of 3 candidate miRs, namely miR-1, miR-25 and miR-613 confirmed their inhibitory effect on the activity of the Wnt pathway. Epistasis experiments revealed that miR-1 and miR-613 target the pathway upstream of Axin or active β-catenin, and that miR-25 acts downstream, at the level of β-cat, likely by targeting β-cat's coding sequence. Importantly, overexpression of miR-25 and miR-1 inhibited proliferation/viability of human colon cancer cells that are known to be dependent on sustained β-cat signaling for their survival[22], [24]. Furthermore, expression of miR-1 in primary mammary epithelial organoids derived from a Wnt-reporter mouse (conductin-lacZ) significantly reduced the expression of the β-gal reporter. These results suggest that these candidate miRs may influence Wnt signaling activity in vivo.

Accordingly, candidate miRs described herein provide novel biomarkers for cancer diagnosis, classification, and prognostic evaluation. Candidate miRs are also envisioned herein as potential therapeutic agents, particularly with respect to disorders relating to the Wnt signaling pathway, such as cancer. Candidate miRs described herein may also be used in screening assays to identify modulators of their activity, which modulators can be used as therapeutic agents for treating disorders relating to aberrant activity of the Wnt signaling pathway.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches. The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program and are known in the art.

The term “functional” as used herein implies that the nucleic or amino acid sequence is functional for the recited assay or purpose.

The term “functional fragment” as used herein implies that the nucleic or amino acid sequence is a portion or subdomain of a full length polypeptide and is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease Si), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

As used herein, the term “operably linked” refers to a regulatory sequence capable of mediating the expression of a coding sequence and which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4, 7, 2′,7′-Tetrachloro-6-carboxyfluorescein (TET). Other alternative DNA labeling methods are known in the art and are contemplated to be within the scope of the invention.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

The compositions containing the molecules or compounds of the invention can be administered for diagnostic and/or therapeutic treatments. In therapeutic applications, compositions are administered to a patient already suffering from a hyperproliferative disorder (such as, e.g., cancer) in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

As used herein, the term “cancer” refers to an abnormal growth of tissue resulting from uncontrolled progressive multiplication of cells. Examples of cancers that can be treated according to a method of the present invention include cancers associated with altered Wnt/wg pathway signaling. Such conditions include a variety of hyperproliferative disorders and cancers, including prostate cancer, breast cancer, skin cancer (e.g., melanoma), colorectal cancer, hepatic cancer (e.g., hepatocellular cancer and hepatoblastoma), head and neck cancer, lung cancer (e.g., non-small cell lung cancer), gastric cancer, mesothelioma, synovial sarcoma, cervical cancer, endometrial ovarian cancer, Wilm's tumor, bladder cancer and leukemia. In a particular embodiment, cancers that can be treated according to a method of the present invention include prostate cancer, breast cancer, skin cancer (e.g., melanoma), and colorectal cancer. In a more particular embodiment, cancers that can be treated according to a method of the present invention include prostate cancer, breast cancer, and skin cancer (e.g., melanoma). See, for example, Luu et al. (2004, Current Cancer Drug Targets 4:653), Lepourcelet et al. (2004, Cancer Cell 5:91), Barker and Clevers (2006, Nature Reviews Drug Discovery 5:997), and Watanabe and Dai (2011, Proc Natl Acad Sci 108:5929), the entire content of each of which is incorporated herein by reference.

As used herein, an “agent”, “candidate compound”, or “test compound” may be used to refer to, for example, nucleic acids (e.g., DNA and RNA), carbohydrates, lipids, proteins, peptides, peptidomimetics, small molecules and other drugs.

The term “control substance”, “control agent”, or “control compound” as used herein refers a molecule that is inert or has no activity relating to an ability to modulate a biological activity. With respect to the present disclosure, such control substances are inert with respect to an ability to modulate a Wnt signaling pathway. Exemplary controls include, but are not limited to, solutions comprising physiological salt concentrations.

The term ‘treating’ or ‘treatment’ of any disease, condition or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease.

The subject is preferably an animal, including but not limited to animals such as mice, rats, cows, pigs, horses, chickens, cats, dogs, etc., and is preferably a mammal, more preferably a primate, and most preferably a human.

The phrase “pharmaceutically acceptable” refers to molecular entities and compositions that are physiologically tolerable and do not typically produce an allergic or similar untoward reaction, such as gastric upset, dizziness and the like, when administered to a human.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All publications mentioned herein are incorporated herein by reference to disclose and describe the methods and/or materials in connection with which the publications are cited.

### Further Aspects of the Detailed Description

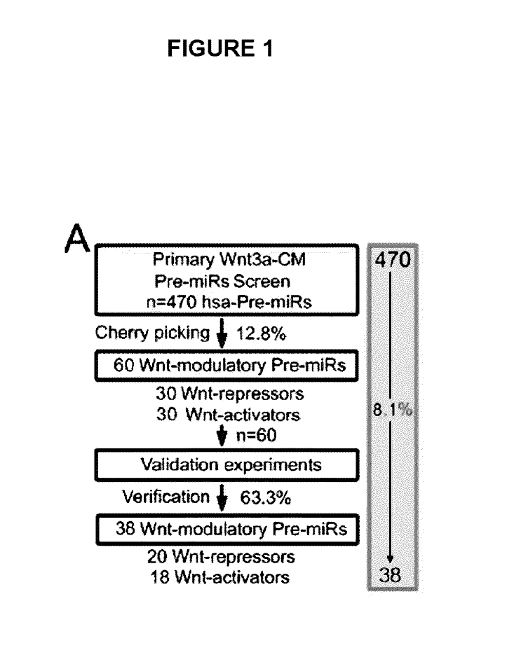

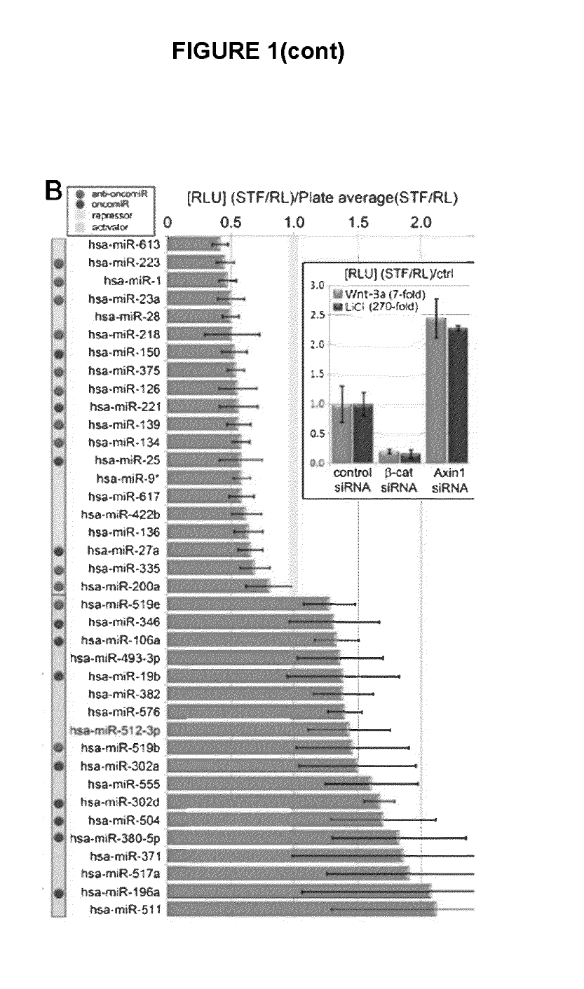

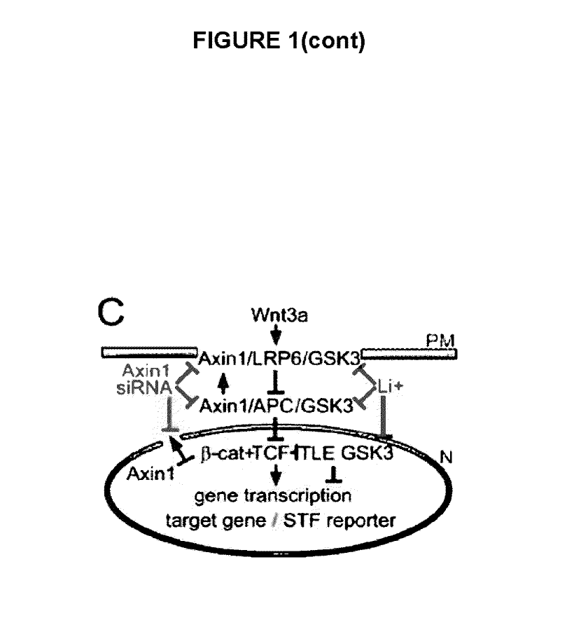

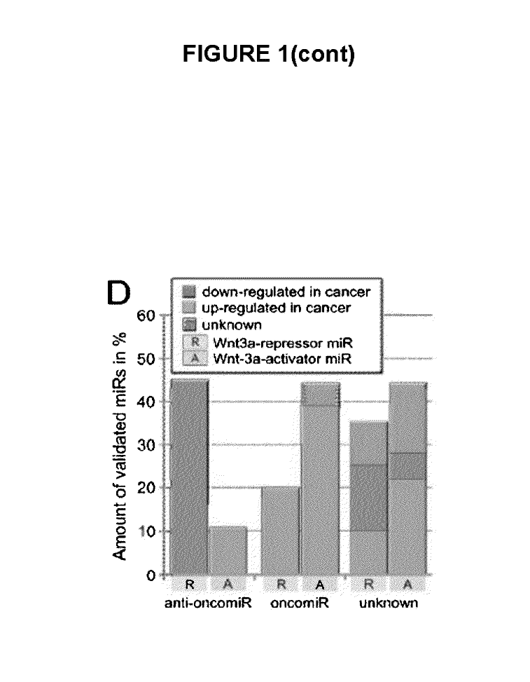

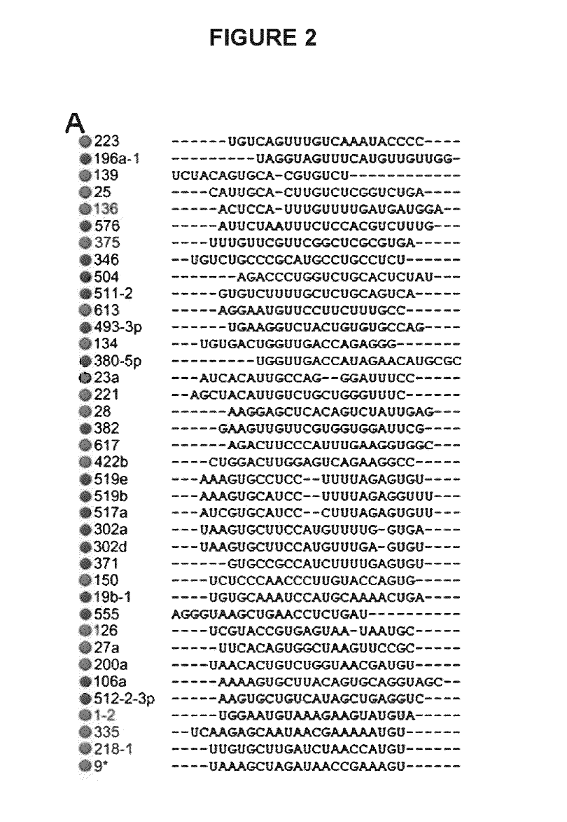

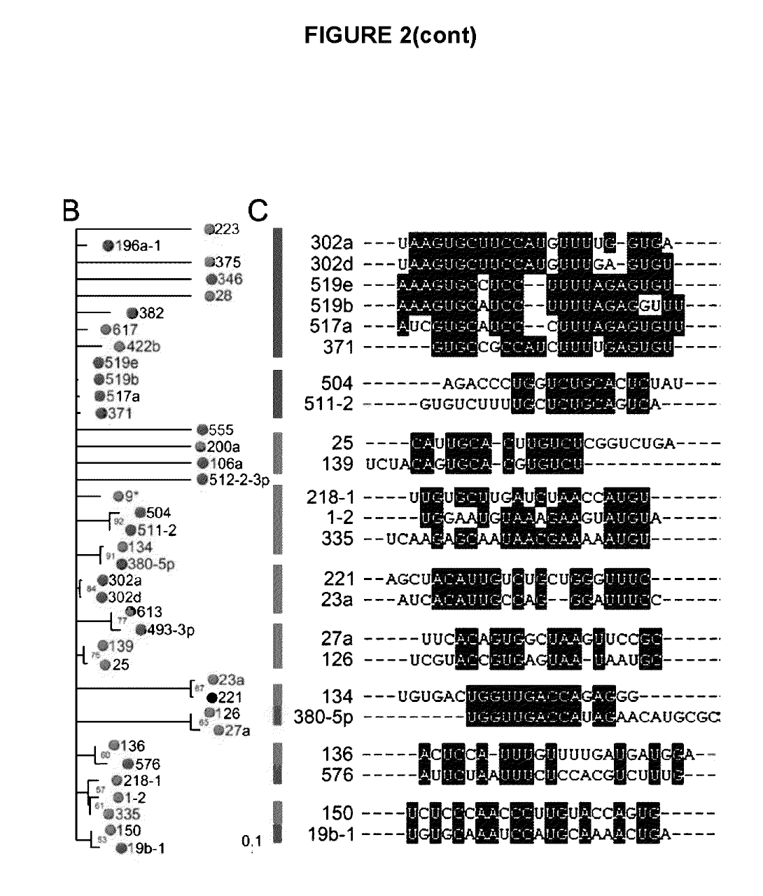

Methods and agents for modulating Wnt signaling pathways in a cell are presented herein. Such agents include the microRNA species listed in FIG. 1 or FIG. 2. MicroRNAs described herein may be used alone or in combination to modulate Wnt signaling pathways in a cell. Modulation of Wnt signaling pathways may be achieved by contacting cells with microRNAs or expressing microRNAs listed in FIG. 1 or 2 in the cell, wherein the contacting or expressing of same modulates expression of at least one nucleic acid sequence encoding a component of the Wnt pathway in the cell. MicroRNAs identified and characterized herein and listed in FIG. 1 or 2 are modulators of Wnt signaling pathways and are, thus, either inhibitors or activators/enhancers of Wnt signaling pathways. MicroRNAs identified as inhibitors of Wnt signaling pathways, for example, are capable of reducing the activity of or inhibiting Wnt signaling pathways, whereas microRNAs identified as activators/enhancers of Wnt signaling pathways, for example, are capable of increasing the activity of or enhancing Wnt signaling pathways.

Exemplary microRNAs identified herein as inhibitors of Wnt signaling pathways and listed in FIGS. 1 and 2 include: microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (also referred to herein as miR-1; SEQ ID NO: 35), 335 (SEQ ID NO: 36), 218-1 (SEQ ID NO: 37), and 9 (SEQ ID NO: 38). Exemplary microRNAs identified herein as activators of Wnt signaling pathways comprise microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), and 512-2-3p (SEQ ID NO: 34).

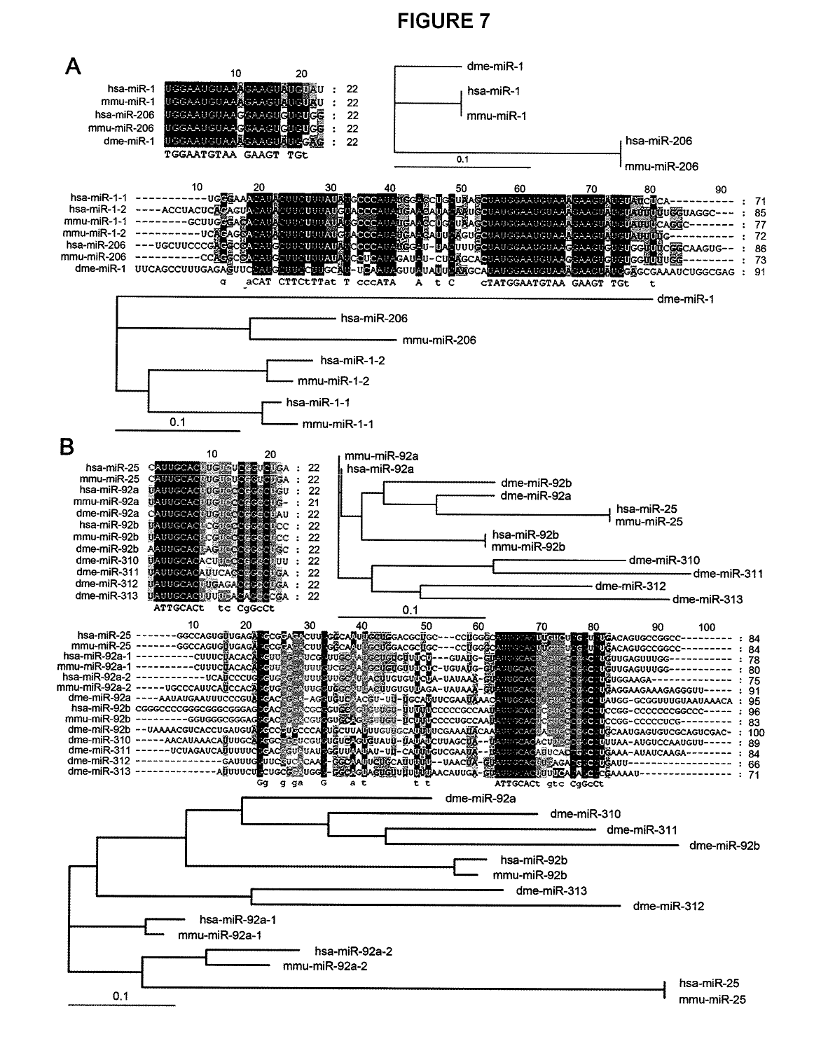

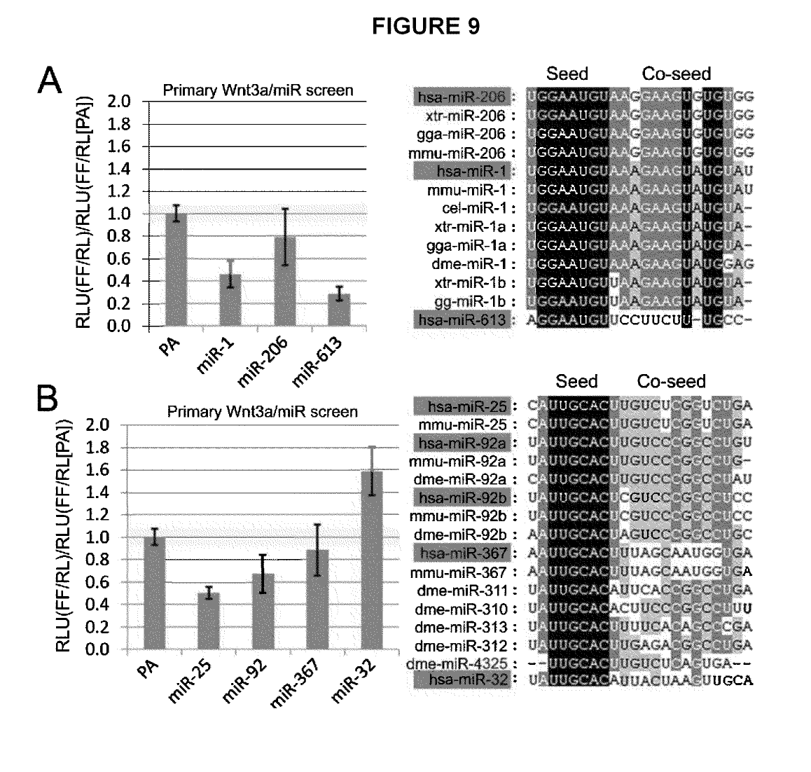

Exemplary microRNAs identified herein as inhibitors of Wnt signaling pathways include members of the miR-1/206 family and the miR-25/92 family. See, for example, FIGS. 7 and 9. Within these families, miR-1 (also referred to herein as miR-1-2), miR-613, miR-25, miR-92a, and miR-92b are particularly exemplary. Inhibitory microRNAs, such as, e.g., miR-1, miR-613, miR-25, miR-92a, and miR-92b, or expression vectors encoding same, may be used to advantage to reduce or inhibit Wnt signaling pathways in cancer cells, wherein the Wnt signaling pathway is elevated or hyper-activated. Under such circumstances, it would be beneficial to reduce or inhibit elevated or hyper-activated Wnt signaling pathways in the cancer cell.

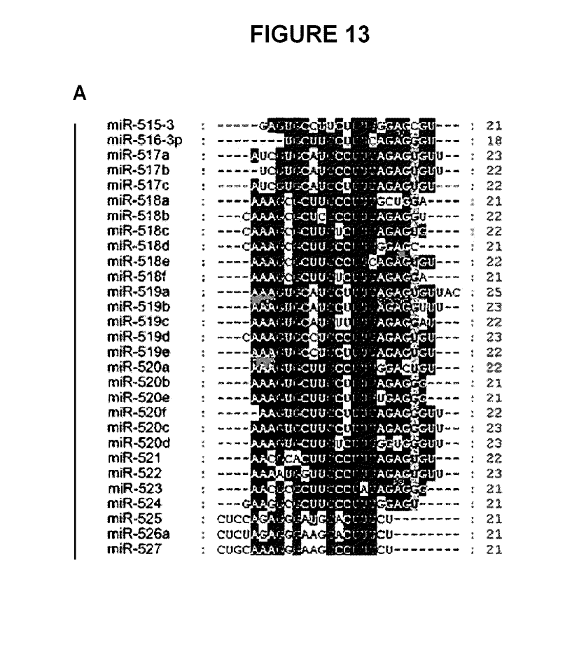

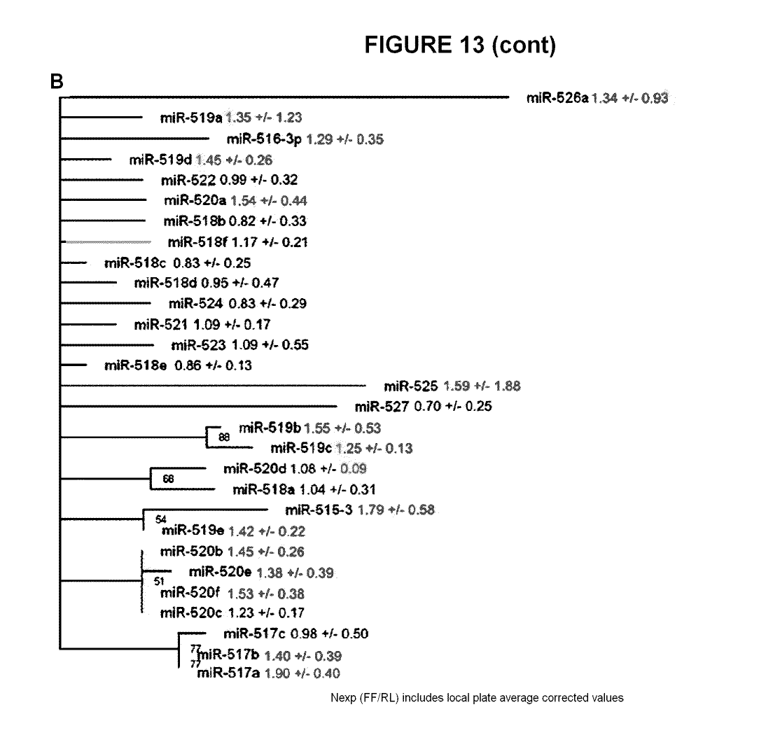

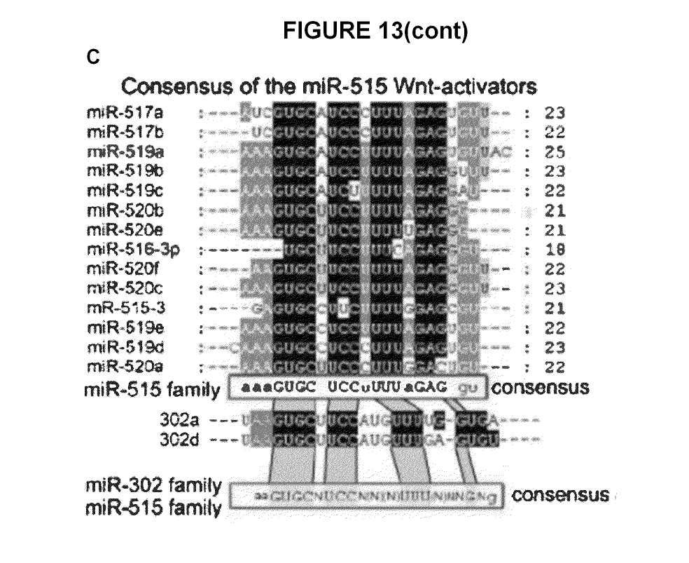

Exemplary microRNAs identified herein as activators/enhancers of Wnt signaling pathways include microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), and 512-2-3p (SEQ ID NO: 34). In a more particular embodiment, the activator/enhancer microRNA is 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In a further embodiment thereof, the activator/enhancer microRNA is a member of the miR-302 family and the miR-515 family. A consensus sequence shared in common among these families is GUGCNUCCN(N)(N)UUU(N)NNGN. See, for example, FIG. 13.

Methods and agents for treating a subject with a cancer associated with an elevated or hyper-activated Wnt signaling pathway are also presented herein. MicroRNAs identified as inhibitors of Wnt signaling pathways have application for such purposes. As indicated herein, microRNAs identified herein as inhibitors of Wnt signaling pathways include members of the miR-1/206 family and the miR-25/92 family. See, for example, FIGS. 7 and 9. Exemplary microRNA inhibitors described herein include, without limitation, miR-1, miR-613, miR-25, miR-92a, and miR-92b. In keeping with guidance presented herein, inhibitory microRNAs, such as, e.g., miR-1, miR-613, miR-25, miR-92a, and miR-92b, or expression vectors encoding same, can be used to reduce or inhibit Wnt signaling pathways in cancer cells associated with elevated or hyper-activated Wnt signaling pathways and thereby treat the subject afflicted with the cancer.

Methods and agents for treating a subject with a bone degenerative disorder (such as osteoporosis) are also presented herein. MicroRNAs identified as activators/enhancers of Wnt signaling pathways have application for such purposes. As indicated herein, microRNAs identified herein as activators/enhancers of Wnt signaling pathways include members of the miR-302 family and the miR-515 family. A consensus sequence shared in common among these families is GUGCNUCCN(N)(N)UUU(N)NNGN. See, for example, FIG. 13.

Methods for expressing microRNAs described herein and expression vectors encoding same are described herein in the Examples section and are, furthermore, known in the art. Exemplary expression vectors include, without limitation, pcDNA3.1(−) vector; retroviral RNAi vectors, including lentiviral vectors such as, e.g., pLV and pCDH-CMV-MSC-EF1-Puro lentiviral vector [See, for example, Yang et al. (Oncogene 22:5694, 2003); Zhou et al. (Oncogene 31:2968-2978, 2012); the entire content of each of which is incorporated herein by reference) and oncoretrovirus vectors; adeno-associated virus (AAV) vectors, particularly those that include a self-complementary genome that enhances therapeutic gene expression and non-human primate AAV serotypes that facilitate efficient transduction following vascular delivery (See, for example, Kota et al. Cell 137:1005, 2009; the entire content of which is incorporated herein by reference); recombinant adenoviral vectors (See, for example, Xia et al. (Nature Biotechnology 20:106, 2002; the entire content of which is incorporated herein by reference); polycistronic RNA polymerase II expression vectors such as those described by Chung et al. (Nucleic Acids Res 34:e53, 2006, the entire content of which is incorporated herein by reference); and tet inducible vector systems. See also Devroe et al. (Expert Opin in Biol Therapy 4:319-327, 2004; the entire content of which is incorporated herein by reference along with references cited therein) for a review expression vectors suitable for delivery of RNAi therapeutics.

Liposomes composed of the neutral lipid 1,2-dioleoyl-sn-glycero-3-phosphatidylcholine (DOPC) have, moreover, been used successfully to deliver an EphA2-targeting siRNA in an orthotopic mouse model of ovarian cancer. Therapeutic delivery of the EphA2-targeting siRNA resulted in decreased EphA2 expression in the tumor and decreased tumor growth when combined with chemotherapy. See, for example, Landen et al. (Cancer Res. 65:6910, 2005); Merritt et al. (J Natl Cancer Inst 100:359, 2008), the entire content of each of which is incorporated herein by reference). In view of the similarities between siRNA and miRNA, it is reasonable to use liposomes composed of DOPC for delivery of the miRNA species desribed herein.

Lipidoids such as those described by Akinc et al. (Nature Biotechnology 26:561-569, 2008; the entire content of which is incorporated herein) for delivery of RNAi therapeutics are also envisioned herein. Akinc et al. demonstrated that the lipidoids described there have broad utility for both local and systemic delivery of RNA therapeutic, both siRNA and miRNA. The safety and efficacy of lipidoids were, moreover, evaluated and confirmed in three animal models: mice, rats and nonhuman primates. Compositions and methods useful for administering nucleic acid based therapies are also described in U.S. Pat. No. 8,034,376, the entire content of which is incorporated herein in its entirety.

Cholesterol conjugation of siRNA has also been demonstrated to improve significantly in vivo pharmacological properties of siRNA. See, for example, Soutschek et al. (Nature 432:173, 2004), the entire content of which is incorporated herein by reference.

Delivery of microRNA may be enhanced by packaging in a nanovesicle or other vehicle developed for microRNA delivery to target tissue in the subject. Nanoparticles have been used successfully for targeted delivery in vitro and in vivo and have, moreover, exhibited reduced toxicity when compared to other therapeutic delivery vehicles. Nanoparticles have been developed for administration via injection and oral consumption. Bisht et al. (Mol Cancer Ther 7:3878, 2008), for example, describe the synthesis and physicochemical characterization of orally bioavailable polymeric nanoparticles composed of N-isopropylacrylamide, methylmethacrylate, and acrylic acid in the molar ratios of 60:20:20 (designated NMA622). Amphiphilic NMA622 nanoparticles show a size distribution of <100 nm (mean diameter of 80+/−34 nm) with low polydispersity and can readily encapsulate a number of poorly water-soluble drugs, including drugs such as rapamycin that are used for treating various cancers, within the hydrophobic core. Mice receiving as much as 500 mg/kg of the orally administered void NMA622 for 4 weeks did not exhibit detectable systemic toxicity. NMA622 nanoparticles, therefore, provide a suitable platform for oral delivery of water-insoluble drugs for cancer therapy and thus, may find application in the delivery of miRNA.

Delivery of RNA therapeutics has also been mediated by direct conjugation of delivery agents to the RNA moiety, formulation using lipid polymer or peptide-based delivery systems and the formation of complexes with antibody fusion proteins. See, for example, Akinc et al. (Nature Biotechnology 26:561-569, 2008) the entire content of which, including references cited therein, is incorporated herein.

Agents described herein, such as, for example, a microRNA may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷CU, ²²⁵Ac, ²¹³Bi, ⁹⁹Tc and ¹⁸⁶Re, which may be attached to agents using conventional chemistry known in the art. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, and urease. Labels further include chemical moieties such as biotin which may be detected via binding to a specific cognate detectable moiety, e.g. labelled avidin. Functional labels include substances which are designed to be targeted to the site of a tumor to facilitate targeted delivery of a microRNA thereto or cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Methods for diagnosing a cancer associated with an elevated or hyper-activated Wnt signaling pathway in a subject are also encompassed herein, wherein such methods comprise determining the level of at least one microRNA shown in either of FIG. 1 or FIG. 2, wherein the at least one microRNA activates the Wnt signaling pathway, and wherein detection of upregulated levels of the at least one microRNA in the subject is diagnostic for the presence of the cancer in the subject. Also described herein are methods for determining the efficacy of a therapeutic agent for treating a cancer associated with an elevated or hyper-activated Wnt signaling pathway in a subject, the method comprising determining the level of at least one microRNA shown in either of FIG. 1 or FIG. 2, wherein the at least one microRNA activates the Wnt signaling pathway, and wherein detection of reduced levels of the at least one microRNA in the subject following administration of the therapeutic agent as compared to prior to administration of the therapeutic agent indicates that the therapeutic agent is efficacious for treating the cancer in the subject. With respect to each of the above methods, in an embodiment thereof the activator/enhancer microRNA detected as a diagnostic or therapeutic indicator/marker is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34). In a more particular embodiment, the activator/enhancer microRNA is 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 302d (SEQ ID NO: 25), 371 (SEQ ID NO: 26), 555 (SEQ ID NO: 29), or 512-2-3p (SEQ ID NO: 34). In a further embodiment, the activator/enhancer microRNA is a member of the miR-302 family or the miR-515 family, which share the consensus sequence GUGCNUCCN(N)(N)UUU(N)NNGN (wherein the nucleotides in bold font are conserved). See, for example, FIG. 13. Methods for detecting microRNA levels are described herein and known in the art.

Methods for determining the efficacy of a therapeutic agent for treating a cancer associated with an elevated or hyper-activated Wnt signaling pathway in a subject are also described, the method comprising determining the level of at least one microRNA shown in either of FIG. 1 or FIG. 2, wherein the at least one microRNA inhibits or represses the Wnt signaling pathway, and wherein detection of increased levels of the at least one microRNA in the subject following administration of the therapeutic agent as compared to prior to administration of the therapeutic agent indicates that the therapeutic agent is efficacious for treating the cancer in the subject. In an embodiment, the inhibitor microRNA detected as a therapeutic indicator/marker is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (also referred to herein as miR-1; SEQ ID NO: 35), 335 (SEQ ID NO: 36), 218-1 (SEQ ID NO: 37), or 9 (SEQ ID NO: 38). In another embodiment, the inhibitor microRNA comprises a consensus sequence as set forth in FIG. 7 or 9. In a further embodiment, the inhibitor microRNA comprises a consensus sequence as set forth in FIG. 7 or 9 and is a member of either of the miR-1/206 family or the miR-25/92 family. In a particular embodiment, the consensus sequence is a GGAAUGU seed sequence. In a particular embodiment thereof, the at least one microRNA is miR-1 or miR-613. In another particular embodiment, the consensus sequence comprises a UUGCAC seed sequence and the microRNA is miR-25, miR-92a, or miR-92b. In an even more particular embodiment, the at least one microRNA is miR-1, miR-25 or miR-613. Methods for detecting microRNA levels are described herein and known in the art.

With regard to methods directed to diagnosis, a sample may be isolated from the subject and analyzed to assess/detect levels of the at least one microRNA. Methods directed to determining the efficacy of a therapeutic agent for treating a cancer associated with an elevated or hyper-activated Wnt signaling pathway in a subject, for example, may be performed using a sample isolated from the subject. Such samples would include fresh frozen or even fixed tissues collected during surgery. Because miRNA are very stable and therefore resistant to degradation, they can be successfully isolated from tumor biopsies and paraffin-fixed tissues.

Identification of particular microRNA that are causally linked with cancers associated with aberrant Wnt signaling will provide information with which a clinician can tailor therapeutic intervention for a patient afflicted with the cancer. If, for example, a patient has a Wnt related cancer wherein an activator/enhancer microRNA described herein or plurality of same is upregulated (expressed at high levels relative to control), then therapeutic intervention can be particularly targeted to decrease levels of this microRNA/s. If, on the other hand, a patient has a Wnt related cancer wherein an inhibitor microRNA described herein or plurality of same is downregulated (expressed at low levels relative to control), then therapeutic intervention can be particularly targeted to increase levels of this microRNA/s. In cases, wherein a patient with a Wnt related cancer exhibits both increased levels of activator/enhancer microRNA described herein and decreased levels of inhibitor microRNA described herein, a skilled practitioner can tailor therapeutic intervention to both decrease levels of the activator/enhancer microRNA and increase levels of the inhibitor microRNA.

The microRNA species described herein may also be used in screening methods to identify a modulators of their activity of at least one microRNA shown in either of FIG. 1 or FIG. 2, the method comprising contacting the at least one microRNA with an agent to determine if the activity of the at least one microRNA is altered in the presence of the agent relative to the activity in the absence of the agent. Potential modulators that may be screened include nucleic acids (e.g., DNA and RNA), carbohydrates, lipids, proteins, peptides, peptidomimetics, small molecules and other drugs.

### Methods of Treatment

The present agents are used as therapeutic agents for the treatment of conditions in mammals that are causally related or attributable to Wnt signaling pathways. Accordingly, the compounds and pharmaceutical compositions of this invention find use as therapeutics for modulating and/or treating a variety of conditions causally related or attributable to Wnt signaling pathways in mammals, including humans. In a particular aspect, microRNAs identified as inhibitors of Wnt signaling pathways are used as therapeutics for treating a cancer associated with an elevated or hyper-activated Wnt signaling pathway. Such cancers include, for example, breast cancer, prostate cancer, colorectal cancer, hepatic cancer, or skin cancer. In an another embodiment, microRNAs identified as activators/enahncers of Wnt signaling pathways are used as therapeutics for treating bone degenerative disorders, such as osteoporosis.

Agents described herein (e.g., micoRNA) may be administered to a patient in need of treatment via any suitable route, including, e.g., intradermal, intramuscular, intravenous, intraperitoneal, intratumoral, oral, rectal, buccal or intranasal administration. The precise dose will depend upon a number of factors, including whether the agents are for diagnosis or for treatment or for prevention. The dosage or dosing regimen of an adult patient may be proportionally adjusted for children and infants, and also adjusted for other administration or other formats, in proportion for example to molecular weight or immune response. Administration or treatments may be repeated at appropriate intervals, at the discretion of the physician.

Agents described herein are generally administered in the form of a pharmaceutical composition, which may comprise at least one component in addition to the agents. Pharmaceutical compositions according to the present invention, and for use in accordance with the present invention, may comprise, in addition to active ingredient, a pharmaceutically acceptable excipient, carrier, buffer, stabilizer or other materials known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, e.g., or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A composition may be administered alone or in combination with other treatments, therapeutics or agents, either simultaneously or sequentially dependent upon the condition to be treated. In addition, the present invention contemplates and includes compositions comprising the agents herein described and other agents or therapeutics such as immune modulators, antibodies, immune cell stimulators, or adjuvants. In addition, the composition may be administered with hormones, such as dexamethasone, immune modulators, such as interleukins, tumor necrosis factor (TNF) or other growth factors, colony stimulating factors, or cytokines which stimulate the immune response and elimination of cancer cells. The composition may also be administered with, or may include combinations along with pathway specific cancer drugs, chemotherapeutics, radiation, and other agents used to treat cancers and known to those skilled in the art.

The preparation of therapeutic compositions which contain agents as described herein and/or polypeptides or analogs as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

Polypeptides for inclusion in compositions can be formulated into a therapeutic composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Other materials as well as processing techniques and the like are set forth in Part 8 of Remington's Pharmaceutical Sciences, 17th edition, 1985, Mack Publishing Company, Easton, Pa., which is incorporated herein by reference.

Accordingly, also encompassed herein is a composition comprising at least one of the miRNA identified herein as an inhibitor of Wnt signaling pathways as listed in FIG. 1 or 2, or nucleic acid sequences encoding same, or an agent identified using a screening assay described herein and a pharmaceutically acceptable buffer, for use in treating a patient with a cancer associated with an elevated or hyper-activated Wnt signaling pathway, such as, e.g., breast cancer, prostate cancer, colorectal cancer, hepatic cancer, or skin cancer, wherein said composition alleviates symptoms of the cancer in the patient when administered to the patient in a therapeutically effective amount. Also encompassed herein is the use of a therapeutically effective amount of a composition comprising at least one of the miRNA identified herein as an inhibitor of Wnt signaling pathways as listed in FIG. 1 or 2, or nucleic acid sequences encoding same, or an agent identified using a screening assay described herein and a pharmaceutically acceptable buffer in the manufacture of a medicament for treating a patient with a cancer associated with an elevated or hyper-activated Wnt signaling pathway, such as, e.g., breast cancer, prostate cancer, colorectal cancer, or hepatic cancer, wherein the medicament alleviates or prevents symptoms of the cancer when administered to the patient. Also encompassed herein is at least one of the miRNA identified herein as an inhibitor of Wnt signaling pathways as listed in FIG. 1 or 2, or nucleic acid sequences encoding same, or an agent identified using a screening assay described herein and compositions thereof for a method of treating cancer in a subject, for use in treating cancer in a subject, and/or in the preparation of a medicament for treating cancer.

Also encompassed herein is a composition comprising at least one of the miRNA identified herein as an activator/enhancer of Wnt signaling pathways as listed in FIG. 1 or 2, or nucleic acid sequences encoding same, or an agent identified using a screening assay described herein and a pharmaceutically acceptable buffer, for use in treating a patient with a bone degenerative disorder, such as, e.g., osteoporosis, wherein said composition alleviates symptoms of the bone degenerative disorder in the patient when administered to the patient in a therapeutically effective amount. Also encompassed herein is the use of a therapeutically effective amount of a composition comprising at least one of the miRNA identified herein as an activator/enhancer of Wnt signaling pathways as listed in FIG. 1 or 2, or nucleic acid sequences encoding same, or an agent identified using a screening assay described herein and a pharmaceutically acceptable buffer in the manufacture of a medicament for treating a patient with a bone degenerative disorder, such as, e.g., osteoporosis, wherein the medicament alleviates or prevents symptoms of the bone degenerative disorder when administered to the patient. Also encompassed herein is at least one of the miRNA identified herein as an activator/enhancer of Wnt signaling pathways as listed in FIG. 1 or 2, or nucleic acid sequences encoding same, or an agent identified using a screening assay described herein and compositions thereof for a method of treating a bone degenerative disorder in a subject, for use in treating a bone degenerative disorder in a subject, and/or in the preparation of a medicament for treating a bone degenerative disorder.

The agent containing compositions are conventionally administered intramuscularly, intravenously, as by injection of a unit dose, or orally, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated and the stage and type of cancer being treated. Precise amounts of active ingredient required to be administered depend on the judgment of the practitioner and are peculiar to each individual. Suitable regimens for initial administration and follow on administration are also variable, and may include an initial administration followed by repeated doses at appropriate intervals by a subsequent injection or other administration.

### Nucleic Acids

The present invention further provides an isolated nucleic acid encoding an agent of the present invention. Nucleic acid includes DNA and RNA.

In a particular aspect, miRNA described herein is synthesized in accordance with standard procedures. miRNA can also be generated via expression constructs in cells, wherein the cell processes the transcript to generate the mature miRNA. Such methods are described in the Examples presented herein and known in the art.

Also envisioned for use in the methods and uses described herein are miRNA mimics, which are small, chemically modified double-stranded RNAs that mimic endogenous miRNAs. In accordance with the methods and uses described herein, an miRNA mimic of an miRNA species identified as an inhibitor of Wnt signaling pathways may, for example, be used to reduce or inhibit Wnt signaling pathways and/or treat a cancer associated with an elevated or hyper-activated Wnt signaling pathway.

In a further application, miRNA inhibitors are envisioned for use in the methods and uses described herein. miRNA inhibitors are small, chemically modified single-stranded RNA molecules designed to specifically bind to and inhibit endogenous miRNA molecules. In accordance with the methods and uses described herein, miRNA inhibitors may, for example, be used to down-regulate the activity of an miRNA species identified as an enhancer of Wnt signaling pathways and thus, reduce or inhibit Wnt signaling pathways and/or treat a cancer associated with an elevated or hyper-activated Wnt signaling pathway.

An exemplary inhibitory molecule of miRNA is an anti-miRNA oligonucleotide (AMO) which blocks the interactions between an miRNA and its target mRNAs by competition. AMOs may be chemically modified in a variety of ways to improve the stability. Locked nucleic acid (LNA), often referred to as inaccessible RNAs, refers to bicyclic high-affinity RNA analogs wherein the ribose moiety is chemically locked in a RNA-mimicking N-type (C3′-endo) conformation by the introduction of an extra 2′-O, 4′-C methylene bridge. The locked ribose conformation enhances base stacking and backbone preorganization and significantly increases the thermal stability of complexes upon hybridization with complementary single-stranded RNA target molecules. In addition, LNA is compatible with RNase H cleavage and displays high aqueous solubility and low toxicity in vivo. The 2′-O-methyl (2′-O-Me) modification, as well as the 2′-O-methoxyethyl (2′-MOE) and 2′-fluoro (2′-F) chemistries are modified at the 2′ position of the sugar moiety and oligonucleotides comprising these modifications have shown promise in functional inhibition of miRNAs. Nuclease resistance is also improved by backbone modification of the parent phosphodiester linkages into phosphorothioate (PS) linkages in which a sulfur atom replaces one of the non-bridging oxygen atoms in the phosphate group or by using morpholino oligomers, in which a six-membered morpholine ring replaces the sugar moiety In addition to chemical modifications, some improvement in inhibitor potency is observed by increasing the length of the AMOs. miRNA sponges, miRNA masking, and small molecule inhibitors are also envisioned herein. See, for example, Li et al. (AAPS J 11:747, 2009; the entire content of which is incorporated herein by reference along with references cited therein) for additional details.

Chemically modified synthetic miRNA mimics and inhibitors are available and commercially available from a variety of vendors. Such vendors include Ambion®, Qiagen, Life Technologies, GenePharma Biotech, and Thermo Fisher Scientific Dharmacon. Thermo Fisher Scientific Dharmacon, for example, can be used as a source for every human, mouse, and rat miRNA present in the miRBase Sequence Database to date (See worldwide web microrna.sanger.ac.uk). Qiagen, for example, can be used as a source of miScript miRNA Mimics, which are chemically synthesized, double-stranded RNAs that mimic mature endogenous miRNAs after transfection into cells. miScript miRNA Mimics are available at cell-culture grade (>90% purity) or animal grade (HPLC purified; for in vivo applications).

In another particular aspect, the present invention provides a nucleic acid which codes for or corresponds a miRNA of the invention as defined above, including any one of those listed in FIG. 1 or FIG. 2 as set out herein.

The present invention also provides constructs in the form of plasmids, vectors, and transcription or expression cassettes which comprise at least one polynucleotide as above. The present invention also provides a recombinant host cell which comprises one or more constructs as above. A nucleic acid encoding any agent as provided herein forms an aspect of the present invention, as does a method of production of the agent which method comprises expression from encoding nucleic acid therefor. Expression may conveniently be achieved by culturing recombinant host cells containing the nucleic acid under appropriate conditions. Following production by expression, an agent may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous miRNA or polypeptides include Chinese hamster ovary cells, HeLa cells, baby hamster kidney cells, HEK293 cells, HCT116, and many others.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. The present invention also provides a method which comprises using a construct as stated above in an expression system in order to express a miRNA as described above.

Another feature of this invention is the expression of DNA sequences contemplated herein, particularly encoding the agents described herein. As is well known in the art, DNA sequences may be expressed by operatively linking them to an expression control sequence in an appropriate expression vector and employing that expression vector to transform an appropriate unicellular host. A wide variety of host/expression vector combinations may be employed in expressing DNA sequences. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences (sequences that control the expression of a DNA sequence operatively linked to it) may be used in these vectors to express DNA sequences. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of the invention. In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products. Considering these and other factors a person skilled in the art will be able to construct a variety of vector/expression control sequence/host combinations that will express the DNA sequences on fermentation or in large scale animal culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Examples

### Methods

Screening and Reporter Assays—

Screening and reporter assays were carried out as described previously [35]. Briefly, HEK293 cells trypsinized and resuspended in antibiotic-free culture media were plated and transfected in 384 well plates (Corning, Cat No. 3704). Transfections were conducted with 0.1 μL Lipofectamine 2000 (Invitrogen) and 27 ng STF19 reporter plasmid (a kind Gift from the R.T. Moon laboratory, Seattle, USA) together with 5 ng Renilla-CMV vector as internal control each well. 7 μL DNA-containing transfection-mix with serum-free DMEM and Lipofectamine2000 was added to the pre-plated Hsa-pre-miR™ in a 384 well plate (5 μL each well, 1.5 pmol, human pre-miR library, n=470, Ambion, #4385830) for a final concentration of 42.9 nM ca. 5000 viable HEK293 cells were plated in 23 μL culture media to the pre-dispensed transfection mixes and incubated for two days. 50 μL of pretested Wnt3a-conditioned media (harvested from L-Wnt3A cells [36], a kind gift from the laboratory or Dr. R. T. Moon, University of Washington, Seattle) were added at day 2 post-transfection and cells were incubated for additional 16 hours. Pre-miRs™ used are synthetic siRNA-like and modified strand selective small dsRNAs with verified specificity (Ambion). Cells were lysed in 20 μL DualGlo (Promega, #E2920) substrate buffer and normalized luminosity, which is the ratio of the firefly reporter (STF16x-Firefly) and the renilla luciferase (CMV-driven), was read with an EnVision multilabel plate-reader (Perkin Elmer). The ratio of the STF-firefly and CMV-renilla RLU (relative light units) for each Pre-miR was divided by the plate average (PA) or control siRNAs, respectively. The primary screen was performed in quadruplets for statistical/assay robustness. Cutoff values were based on the performance of control siRNAs with the only exception of the high/medium-scorer miR-200a that was also included into the cherrypick-listing because of its described role[29], [32]. Raw data, normalized values, and cherry-picking listings are deposited available at the NYU-RNAi core facility and available upon request. Other reporter assays, including epistasis experiments, were conducted in 96- or 384-well plates using the same protocol settings. STF19/CMV-RL activity in cancer cell lines was measured one day post-transfection.

Cloning, DNA and RNA Reagents

The coding region of human β-catenin (S37A mutant; transcript nt position 307-1874 NcoI-Klenow; NotI) was subcloned into the 3′UTR of the Renilla gene (NotI, SpeI-Klenow) within a psi-check-2 reporter vector with modified MCS to monitor the influence of miR-25 on its transcript stability and translation. In this assay a CMV-driven synthetic-firefly-luciferase served as internal control. A β-catenin fragment lacking exon-3 (543-1874) behaved similar (not shown). A human Pri-miR-25 fragment was PCR amplified from human genomic DNA (HEK293 cells) with the following primer pair: FP-miR-25: 5′-gcggccgccattctcagacgtgcctaag-3′, RP-miR-25: 5′-tctagatgattacc-caacctactgct-3′. After sub-cloning the hsa-Pri-miR-25 amplicon that contains endogenous 5′- and 3′-flanking sequences was finally cloned into the pcDNA3.1(−) vector (Invitrogen) via BamHI (vector and insert) and NheI (vector) XbaI (insert). HPLC grade human synthetic Pre-miR™ precursor miRNAs that are strand-selection optimized/approved and chemically modified siRNA-like precursor miRs (Pre-miR-1™ #AM17100; Pre-miR-25™ #AM17100; Pre-miR-613™ #AM17100) were purchased from Ambion. Three Axin1 and Axin2 Stealth siRNA (Invitrogen, Cat. No.: 119026-B03, -B05, -B01, -007, -C11, -009) were used as positive control and epistatic-inductors to activate the Wnt pathway at its downstream components. Three Stealth siRNAs for β-catenin (Invitrogen, 119026B07/B09/B11) were used to down-regulated Wnt activity as positive control for a Wnt-pathway inhibitory RNA. Silencer-Negative Control siRNA #4 (Ambion, Invitrogen #AS00K2L1) and Silencer-Negative control siRNA #2 (Ambion, Invitrogen #AS00JSIG) were used as negative controls. The Pre-miR™ precursor molecule library was plated in 5 μL with 1.5 pmol each well into two 384 well plates at the RNAi screening core facility using an automated dispenser system (Janus MDT, Perkin Elmer) and stored at −80° C. pLV-miR-1 and pLV-miR-control lentivirus were obtained from Biosettia (San Diego, USA).

Bioinformatics, and Statistical Analysis

Sequences were aligned with ClustalW, visualized and processed with Genedoc software, and Treepuzzle and Treeview software were employed for similarity and phylogenetic analysis (parameters: 10.000 puzzling steps; quartet puzzling tree reconstruction, neighbor-joining tree, approximate quartet likelihood, HKY-model of substitution). miRNA target prediction and alignment blasts were done with miRWalk, Pictar, Diana-microT, PITA, RNAhybrid, Target Scan, miRanda, NCBI-blasts. Statistical analysis: Z-factors were calculated with Z=1−(3(σₚ+σₙ)/(|μₚ−μₙ|)) and values between 0.5 and 1.0 indicated good screening parameters. Log-Z-score was deployed on log-tansformed (Nexp FF/RL) data set. Z=((FF/RL)_{LOG}−PA_{LOG})/STDEV(P_{LOG}); FF, TCF19x-firefly RLU; RL, CMV-Renilla RLU; PA, plate average; RLU, relative light units; STDEV(P) standard deviation of the plate. Local plate averages (7-10 data points) were used for alternative balancing of normalized data of the primary screen. Regulation of transcript level/translation is measured by the psi-check-2 reporter and determined by relative changes of RLU values via R(r)=(RL−β−cat_{CDS}/FF)/(RL−empty/FF); with R(r) of controls=1. Pubmed (NCBI) and Google searches were used for data mining to find cancer relevant reports on miRs identified and verified for correlation studies.

Western Blotting

SDS-PAGE and western blotting was performed with standard protocols including TBST (0.1%) washing buffer and 4% BSA TBST blocking buffer. Primary antibodies (anti-β-catenin, Sigma, #C7207, anti-tubulin (α-Tubulin), Sigma, #T9026) were incubated over night while gently shaking at 4° C. in 1:1000 to 1:2000 dilutions. Infrared (IR)-dye conjugated secondary antibodies (1:20.000, goat anti-mouse, IRDye™800, #610-132-121, Rockland) were incubated at room temperature for 1 h in blocking buffer and subsequently rinsed with TBST. Blots were visualized with the Odyssey infrared imaging system and quantified with the Odyssey software (Li-Cor, Biosciences). For semi-quantitative western blotting the average of the ratios of β-catenin and α-Tubulin (α-TUB) [signal intensity] was divided by the average of the ratios of the control experiments.

Cell Culture and Cell Lines

HEK293 human embryonic kidney cells (ATCC, cat # CRL-1573), HCT116 human colon cancer cells (ATCC, cat # CCL-247), SW480 human colon cancer cells (ATCC, cat # CCL-228), and MCF7 human breast cancer cells (ATCC, cat # HTB-22) were cultured in filtered DMEM media supplemented with 10% fetal calf serum, 1 mM L-glutamine and 1× non-essential amino acids (ATCC, #203166) without antibiotics at 37° C. and 5% CO₂. Selection media for cells transfected with linearized empty pcDNA3.1(−) or Pri-hsa-miR-25 pcDNA3.1(−) using Lipofectamine2000 contained increasing amounts of active G418 sulfate (Cellgro #30-234-CR) for 7-16 days. Lenti-pLV-miR-1 and -pLV-control transduced cells were selected with puromycin for up to 7 days.

RT-qPCR Real-Time relative quantification PCR was conducted with 25 μL iTAQ™ SYBR-Green Supermix (lx) with ROX (BIO-RAD, #20361) and the qPCR thermocycler Mx3005P (Stratagene) system including the MxPro-Mx30055 v.4.10 Build 389 software (Stratagene) and the delta-delta-Cᵣ calculation method. Thermocycler conditions: 96° C. initial denaturation step for 10 min, 50 cycles of (96° C. denaturation for 30 s, 57° C. annealing for 30 s and 72° C. elongation for 10 s). Amplification and dissociation curves revealed the specificity of the qPCR products, which were also examined by DNA agarose TAE gel electrophoresis (2% agarose, with EtBr). Unmodified exon-spanning primer pairs with similar annealing temperature (57° C.) and product size (80-110 bp) were used. Total RNA was isolated with RNeasy (Qiagen) and cDNA was synthesized using the High-capacity cDNA reverse transcription kit (Applied Biosystems, #4368814). Briefly, 1 μg of total RNA was DNaseI digested, heat inactivated, and input for cDNA reverse transcription in 20 μL following the instructions of the manufacturer. Primer sequences: CTNNB1-fw 5′-ATGGCAACCAAGAAAGCAAG-3′; CTNNB1-ry 5′-GGTCCACAGTAGTTTTTCGTAAG-3 (product size 101 bp); internal control primer: GAPDH-fw 5′-TGAAGGTCGGAGTCAACG-3′, GAPDH-ry 5′-GGGTCATTGATGGCAACA-3′ (product size 97 bp).

In Vivo Context Analysis of the Regulation of Axin2/Conductin-lacZ Reporter by miR-1:

Two axin2/Conductin-lacZ mature female mice [37] were sacrificed and mammary epithelial organoids prepared essentially as described [38]. Six mammary glands/mouse were collected in DMEM/F12 10% FBS medium on ice. The glands were transferred to a sterile Petri dish and minced into a homogenous paste using a pair of sterile scalpels. The minced tissue was transferred to a 15 ml falcon tube containing 9 ml Epicult-B Basal Medium (Stem Cell Technologies) and 1 ml of 10× collagenase and hyaluronidase (3000 and 1000 units/ml Stem Cell Technology) and constantly agitated (environmental shaker) for 1 h at 37° C. The tissue was then collected by centrifuged at 450 g for 10 min and re-suspended in cold HBSS 2% FBS. The organoids were isolated from contaminating fibroblasts and blood cells by 2 s pulse centrifugation at 450 g (repeated 5-7 times) their purity was assessed by examination on a glass slide. The final pellet was re-suspended in 2 ml Trypsin/EDTA (0.25%) and incubated at 37° C. for 1 min. The sample was then diluted in 10 ml of cold HBSS containing 2% FBS and spun down at 450 g for 5 min. The supernatant containing stringy DNA was removed carefully and the remaining pellet was re-suspended in pre-warmed fresh dispase containing 500 μl (2000 unit/ml) DNase, mixed for 1 min and spun down. Organoids were then re-suspended and plated in Epicult-B Basal Medium. Control and miR-1 expression vectors (pLV-miR-1 from Biosettia Inc., USA) were used for lentiviral transduction of the cells in 24-well plates. After 12 h of incubation, the cells were fixed in 4% formaldehyde (Electron Microscopy Sciences) for 20 min, washed 3 times with 1×PBS, permeabilized and blocked with blocking buffer (0.1% Triton X-100, 1% BSA, 5% normal goat serum in DPBS) at room temperature for 20 min and immuno-stained with mouse anti-β-GAL 1:50 (DHSB, 4° C. overnight), anti-mouse IgG-Alexa488 goat antibody (Invitrogen; 1:1000; 2 h at room temperature) and DAPI. The cells were imaged using a Nikon TE2000PFS and the levels of β-gal (fluorescence intensity) was measured by the NIS elements software (Nikon, USA).

### Results

Identification of Human microRNAs in a Primary Wnt-Reporter Screen

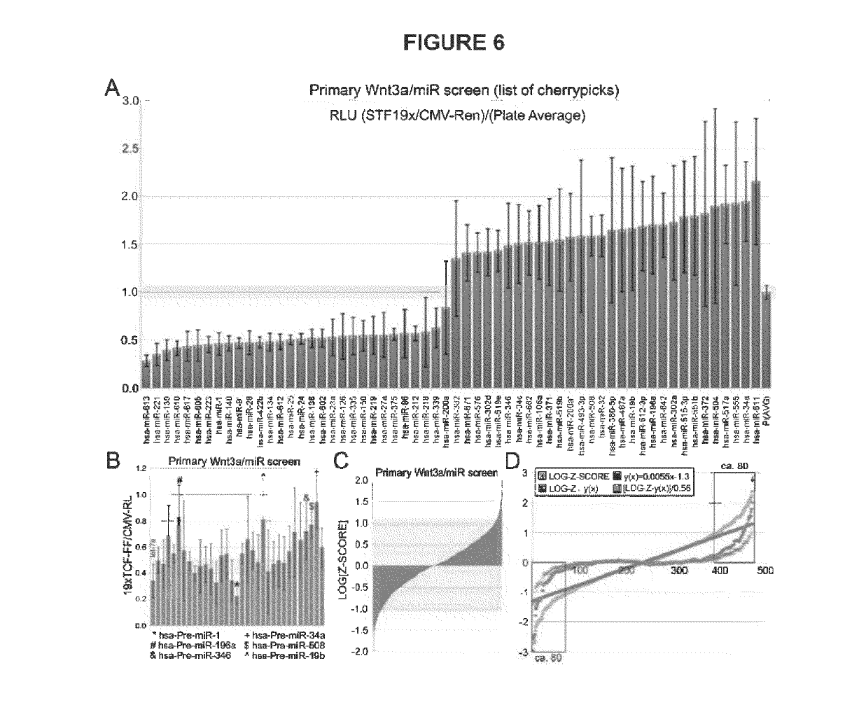

Micro-RNAs (miRNAs, miRs) and the Wnt pathway play fundamental roles in development and disease by regulating expression of target genes. We hypothesized that certain miRs could fine-tune the activity of β-catenin-dependent Wnt signaling by modulating the abundance of relevant pathway components. To address this question we performed a luciferase reporter screen in human HEK293 cells to investigate the regulatory capacity of a library of 470 miRs on the activity of the Wnt pathway. HEK293 cells were transiently co-transfected with individual synthetic Pre-miR™s, a luciferase gene reporter driven by 16 functional TCF/LEF1 binding sites and a CMV-Renilla luciferase that served as internal control for cell viability and transfection efficiency. The Wnt pathway was activated with Wnt3a-conditioned media from L-cells 2.5 days post-transfection, and plates were read after 16 h of Wnt3a-CM treatment at day 3. The primary screening was performed in quadruplets in a 384-well plate format and yielded a set of 60 (of 470) candidate Wnt-regulatory human miRNAs (30 activators/synergists and 30 repressors), representing 12.8% of the strand-selective, synthetic Pre-miR™ Library (Ambion) (FIG. 1A, FIG. 6). A sector of the averaged and normalized screening results and a representation of the Z-score of the entire log-transformed data set is shown in FIG. 6 (B, C). The pCMV-Renilla-normalized Wnt/miR-screening results are available. Following verification of consistent “hit” reproducibility, in a second round of low-throughput screening, 38 of the original 60 Pre-miRs were validated as unidirectional regulators, representing a validation rate of 63.3% which is about 8% of the entire library (FIG. 1A, B). Positive controls with β-catenin siRNAs, Axin1/Axin2 siRNA, and negative siRNA controls showed expected and reproducible results (FIG. 1 B, inset). The z-factor for the high throughput screen (HTS) was determined to be z>0.55 suggesting a robust setup for HTS and was also utilized for cut-off designations together with the normalized values of all positive controls.

Wnt-Modulation by miRs Correlates with miR-Oncogenicity and Sequence Similarity.

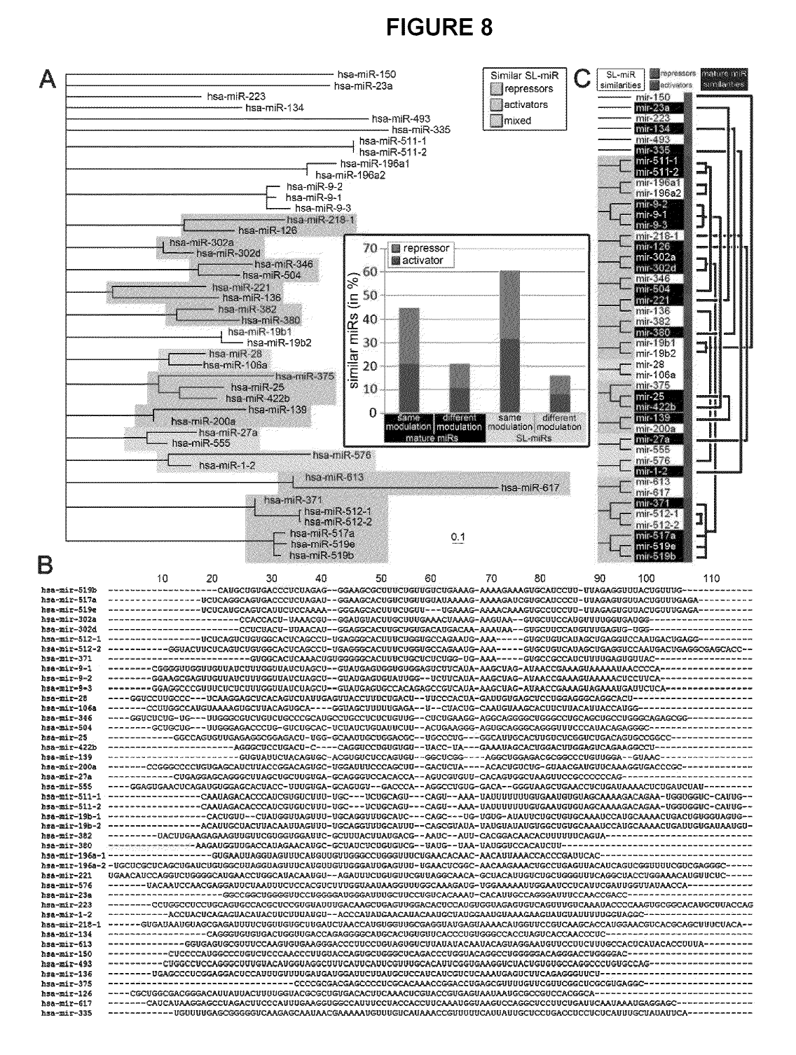

Having determined the initial list of verified Wnt-modulating miRs (FIG. 1B), we examined possible correlations between the candidate miRs. An alignment of all mature miRs revealed a significant enrichment of sequence similarities within all validated miRs, some of which are members of the same family. Notably, we found that sequence similarity between miRs correlates with their ability to either activate or inhibit the activity of the Wnt-pathway (FIG. 2). An unbiased alignment of all identified mature miR sequences, shown in FIG. 2A, indicates several obvious consensus sequence conservations amongst all validated candidate miRs. MiRs with high sequence similarity can be also easily identified in a quartette puzzling tree, FIG. 2B, where nucleotide substitution is a measure of branch length like in phylogenetic trees. Wnt-repressing miRs are indicated by a green disc and Wnt-activators are indicated by a red disc. Thus it became apparent that high sequence similarity often goes in parallel with unidirectional modulation of the Wnt pathway (FIG. 2B). Most obvious and interesting sequence similarity examples are also shown in FIG. 2C. Specifically, this correlation or trend was very clear for 6 miRs, miR-302a, -302d, -519e, -519b, -517a, and -371 e.g.; they all hyper-activated the Wnt-reporter and happen to exhibit high sequence similarity (FIG. 2C). Of these, miR-302a and miR-302d are members of the miR-302 family and miR-519e, -519b, and -517a belong to the miR-515 family. A closer look at sequence similarity analysis of the whole miR-515 family revealed a striking consensus sequence between related Wnt-activating miRs (FIG. 13). Moreover, this consensus sequence is also highly shared by the miR-activators of the miR-302 family. Hsa-miR-371 is part of the miR-290 family, in agreement with alignments and phylogenetic Stem Loop (SL)-miR trees (FIG. 8), but also inherits, what we would suggest, a newly identified “Wnt-activator associated signature” (GUGCNNCCN(N)(N)UUU(N)NNG). Curiously, this activator consensus sequence is discontinuous and consists of the 3′-half of the major and highly conserved miR-seed sequence and a more 5′-part of a “co-seed” sequence, which is part of the mature miR and 3′-downstream of the major-seed. This consensus sequence could thus play a role in target mRNA recognition in addition to its putative function in the formation of the loop structure of the stem-loop miR precursor. A comparison of Pre-miR-SL and mature miR sequence alignments and trees also helps to discern conserved versus non-conserved/adopted consensus and seed sequences (FIG. 8). Importantly, the majority of these similar mature miRs and SL-miRs shows a modulation of the Wnt pathway in the same direction (FIG. 8, inset), which could indicate a causal relationship between sequence similarity/specificity and similar function.

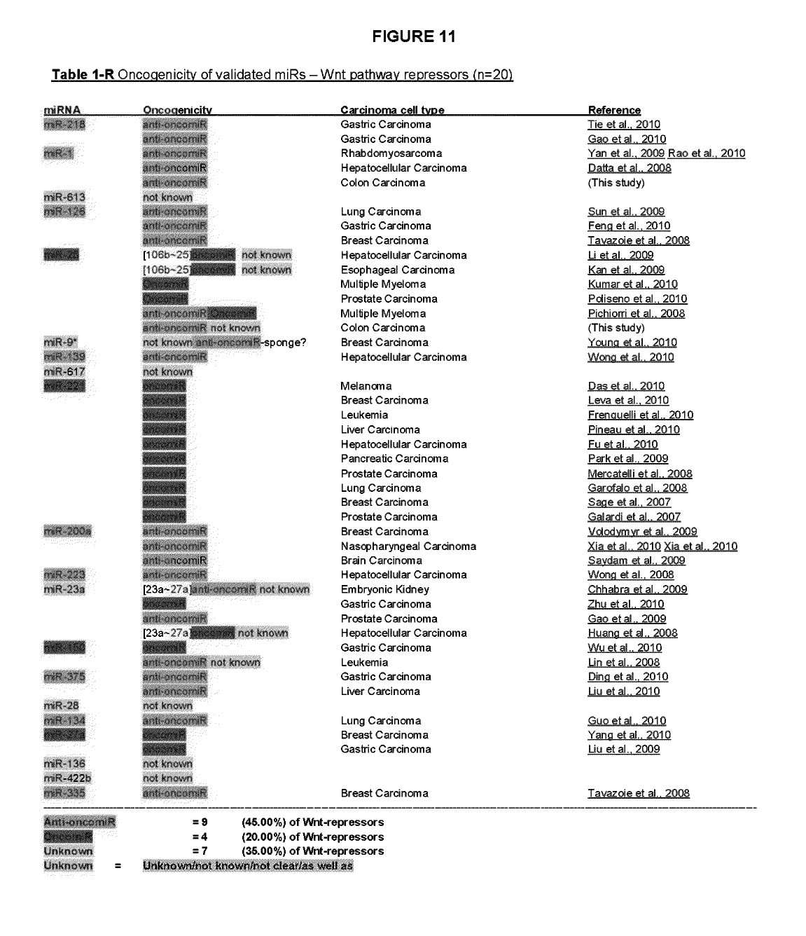

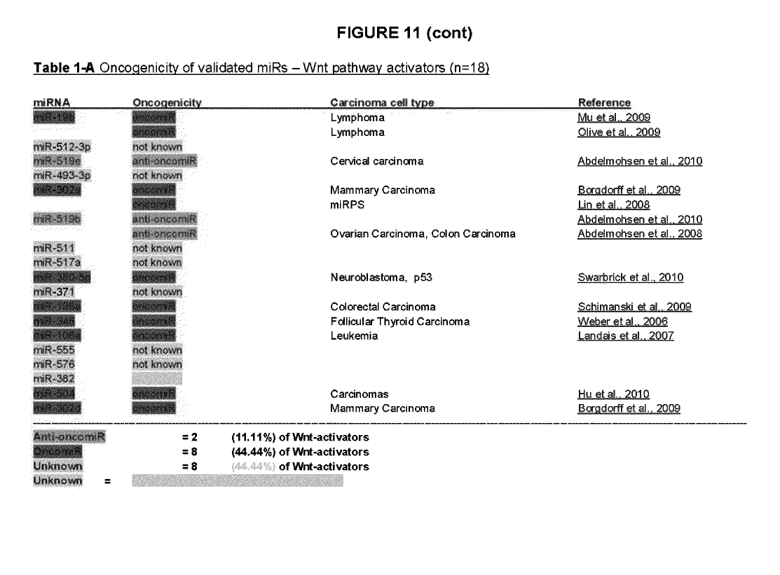

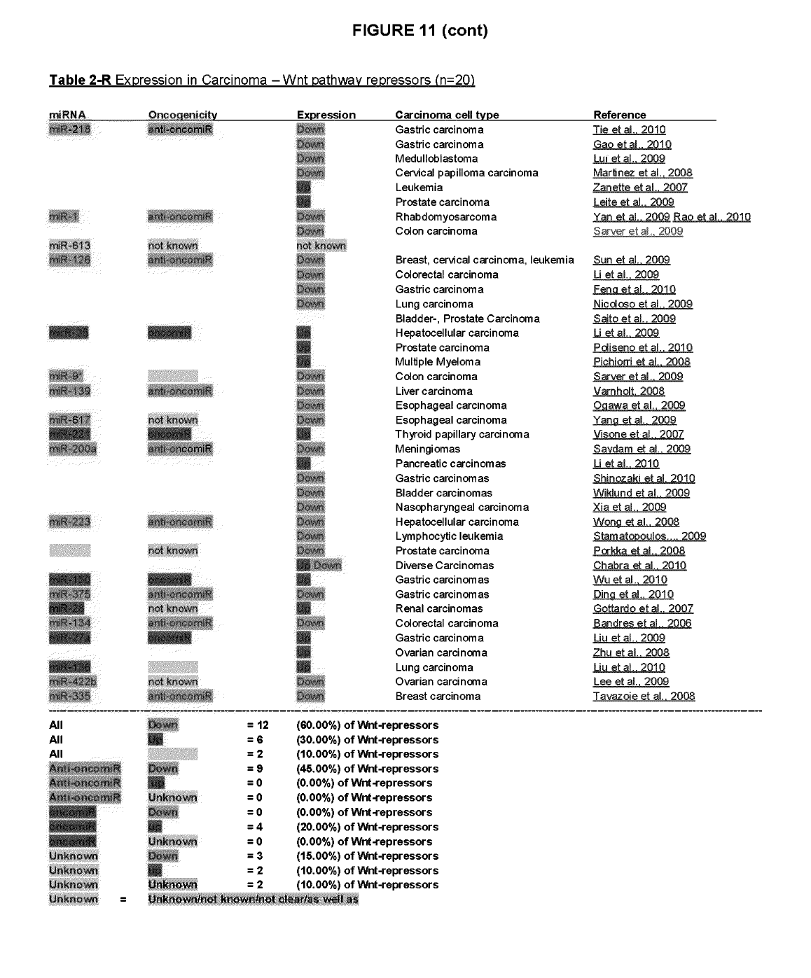

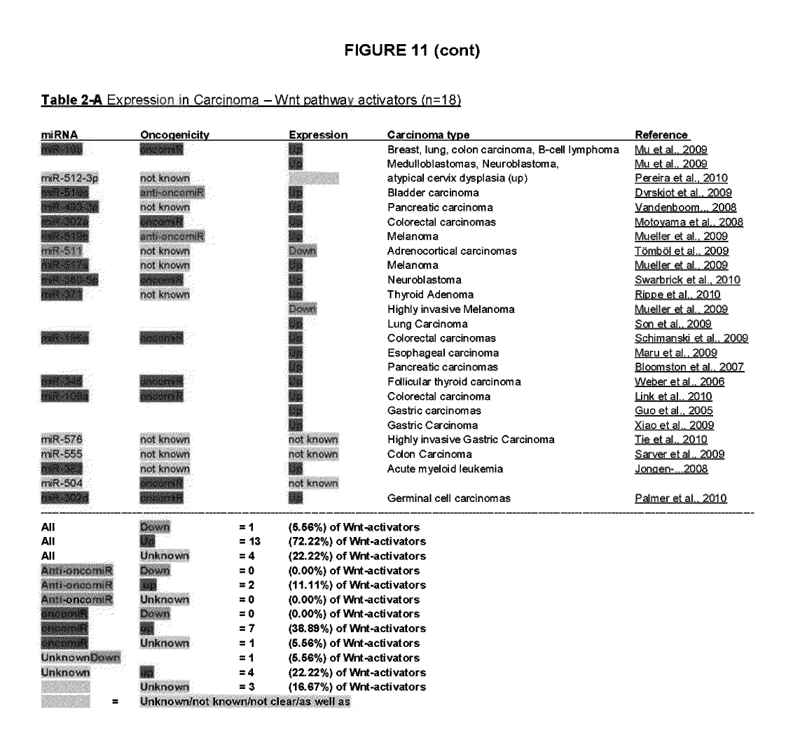

Interestingly, further data mining via literature searches revealed another outstanding and surprising coherency that also supported our initial hypothesis: a majority of the miRs that have been reported to be anti-oncogenic and are repressed in cancers displayed Wnt-inhibitory properties, whereas those described as oncogenic miRs and are often elevated in cancers were identified as activators of the Wnt pathway by trend (FIG. 1D, for references see Table in FIG. 11).

Secondary Validation of miR-1, miR-25 and miR-613.

We chose to further investigate three candidate miRs that repressed Wnt3a/β-catenin signaling in the primary screen: miR-1, miR-25 and miR-613. Phylogenetic analysis support the miR-base classification that miR-1 belongs to the miR-1/206 family including hsa-miR-206 and the Drosophila dme-miR-1, an indication of the high evolutionary conservation of this family (shown in FIG. 7A by alignments and phylogenetic quartette puzzling trees); hsa-miR-25 belongs to the evolutionary conserved miR-25/92 family [39] including Drosophila miR-92a+b/310/311/312/313, shown in FIG. 7B, and shares only the seed sequence with other miRs like miR-4325 or miR-367. MiR-613 showed the strongest inhibition in the primary validation assay (FIG. 6) and shares the same seed sequence as the miR-1/206 superfamily (5′-GGAAUGU-3′) but shows no additional overall conservation (FIG. 9). On the other hand miRs like miR-32 or 367 that shares the seed with the miR-25/92 family could not repress Wnt reporter activity (FIG. 9). This could indicate that some miRs or miR-families may repress the Wnt pathway components/activity mainly with the seed sequence (miR-1/613-family), while others may require the coordinated action of the seed and co-seed (miR-25/92-family).

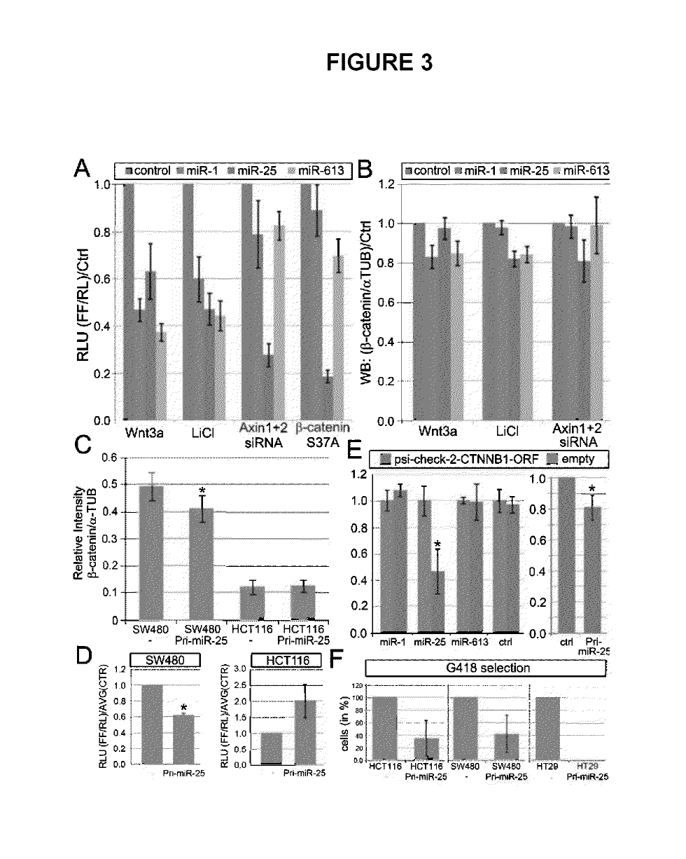

To understand at which step these miRs (miR-1, -25, -613) modulate the linear cascade of the Wnt pathway, and to identify their potential target genes, a series of epistasis experiments were conducted in HEK293 cells using the Wnt reporter and different pathway activators (FIG. 3 A, B). Induction of the Wnt pathway with Wnt3a-CM or by the GSK3β-inhibitor Lithium salt (LiCl) could be repressed by all three miRs. Elevated reporter activity by simultaneous siRNA mediated knockdown of Axin1 and Axin2 could be strongly inhibited by transfection of Pre-miR-25 (P<0.05; unpaired t-test), while miR-1 and miR-613 showed no significant influences (P>0.05). While Axin1 and GSK3β occupy similar complexes at the plasma membrane with LRP6, and with β-catenin in the cytosolic destruction complex, their functions may diverge elsewhere, such as in the nucleus where Axin-1 is also thought to be involved in nuclear export mediated repression of β-catenin function[40], [41]. These observations suggest an additional role of Axin downstream/independent of GSK3β. In agreement with this, Axin1/2 siRNAs could further synergize pathway activation in Wnt3a (7-fold activity) and LiCl (270-fold activity) induced cells to a very similar extent (FIG. 1 B inset), indicating that Axin inhibition, activates the pathway additively irrespective of the level of GSK3β-inhibition. Additionally, only miR-25 inhibited the activity of degradation-resistant S37A β-catenin mutant on the STF reporter (FIG. 3 A). Taken together, these data suggest that miR-25 represses the Wnt pathway downstream of GSK3β, Axin1/2 and stabilized β-catenin, while miR-1 and miR-613 act upstream of Axin1/2 and stabilized β-catenin but probably downstream of LiCl-mediated inhibition of GSK3|3.

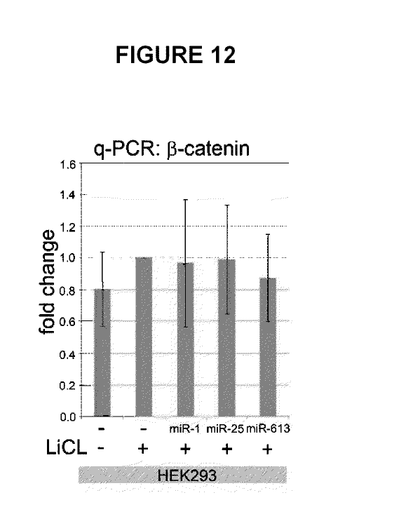

Relative quantification with real time quantitative PCR (RT-qPCR) did not reveal any significant reduction in β-catenin (CTNNB1) mRNA level in HEK293 cells (FIG. 12), nor for BCL9, Lef-1, TCF-1 (TCF7), Pygo1, Pygo2, Mamll, TCF-3, (TCF711) TCF-4 (TCF712) and western blotting also revealed no change for TCF-4 (not shown). While miR-1 and miR-613 could slightly reduce Wnt3a-CM mediated induction of β-catenin protein levels in HEK293 cells, miR-25 and miR-613 expression resulted in a moderate (˜20%) reduction in LiCl induced total β-catenin protein level, (FIG. 3B).

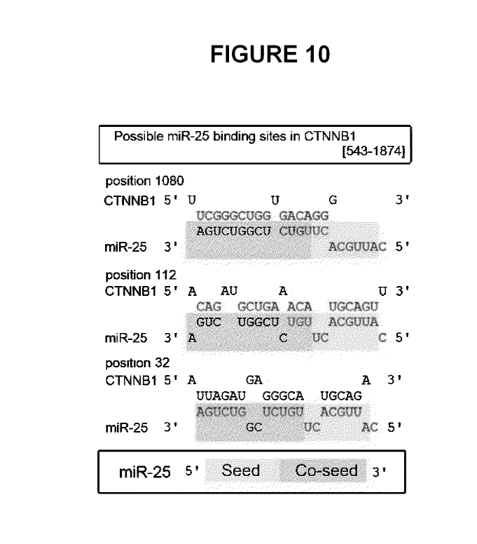

The epistasis experiments indicated that miR-25 may act in parallel or downstream of β-catenin itself. Intriguingly the most stringent RNAhybrid predictions that allow non-canonical seed sequences indicated some potential binding sites of miR-25 in the β-catenin cDNA (FIG. 10), while other target prediction algorithms (PicTar, EMBL-Microcosm, etc.) did not predict β-catenin as a target, because there is no distinct site in its 3′UTR. To test whether miR-25 could directly target the β-catenin cDNA, a fragment of the β-catenin coding sequence (CDS) containing the potential miR-25 binding sites was cloned into the psi-check-2 reporter. Renilla gene activity with an inserted β-catenin CDS in the 3′UTR indicated a significant miR-25-dependent reduction while control siRNAs, miR-1 or miR-613 had no effect (FIG. 3E). Notably, not only synthetic Pre-miR-25 but also over-expression of Pri-miR-25 could reduce a normalized Renilla gene activity with the β-catenin-CDS-fragment-3′UTR but to a lesser degree. As RT-qPCR experiments revealed no significant change in β-catenin transcript levels for all three miRs (FIG. 12), one remaining possibility is that miR-25 represses translation of β-catenin but not its transcript level. As β-catenin is predominantly and tightly regulated via post-translational regulation it is likely that translational reduction might play a minor role in uninduced or moderately induced cells, such as by Wnt3a-CM which induces reporter activity by only 7-10-fold (FIG. 3B); while during extensive pathway activation via LiCl, which can induce a >200-fold induction in the STF-assay, translational differences could play an increasingly relevant and important function in modulating the steady-state protein levels and are thus then detectable in our assays.

Investigating Functions of miR-1 and miR-25 in Wnt-Relevant Colon Cancer Cell Lines.

In order to investigate the function of miR-25 in Wnt-responsive cell lines we cloned a human unprocessed Pri-miR-25 into the pcDNA3.1(−) expression vector with a selectable neomycin marker. Upon transfection of colon cancer cells (HCT116, HT29, SW480) with Pri-miR-25 expressing vector, the number of Pri-miR-25 stable cell-colonies was markedly reduced compared to empty vector controls (FIG. 3F). This was particularly evident in HT-29 cells where despite several attempts we could not generate HT29-pcDNA3.1(−)-hsa-miR-25 expressing stable cell lines, although a few pcDNA3.1(−) empty vector transfected clones survived the selection procedure (FIG. 3F). HT29 cells are known to be dependent on APC-deficiency induced β-catenin activity for their survival, as reintroduction of a cDNA coding for a wild-type APC induces apoptosis [22], which could explain our observation. Additionally, expression of Pri-miR-25 in SW480 colon cancer cells, that exhibit high Wnt/β-catenin activity due to an APC truncation, significantly inhibited both the STF Wnt-reporter activity by ˜40% (FIG. 3D) and β-catenin protein levels by 20% (FIG. 3C). However HCT116 colon cancer cells that are known to exhibit high levels of endogenous Wnt pathway activity due to an intrinsic mutation in the β-catenin gene (AS45) showed no reduction in the STF and β-catenin expression levels.

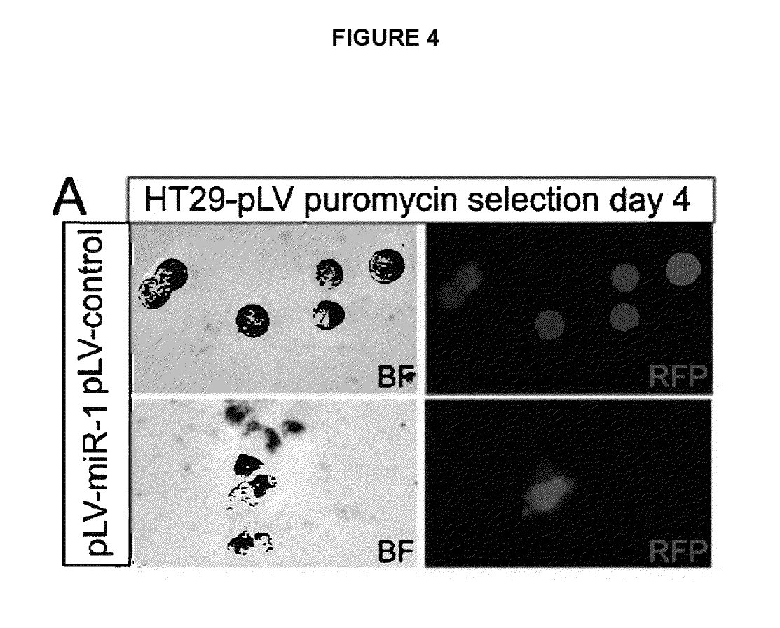

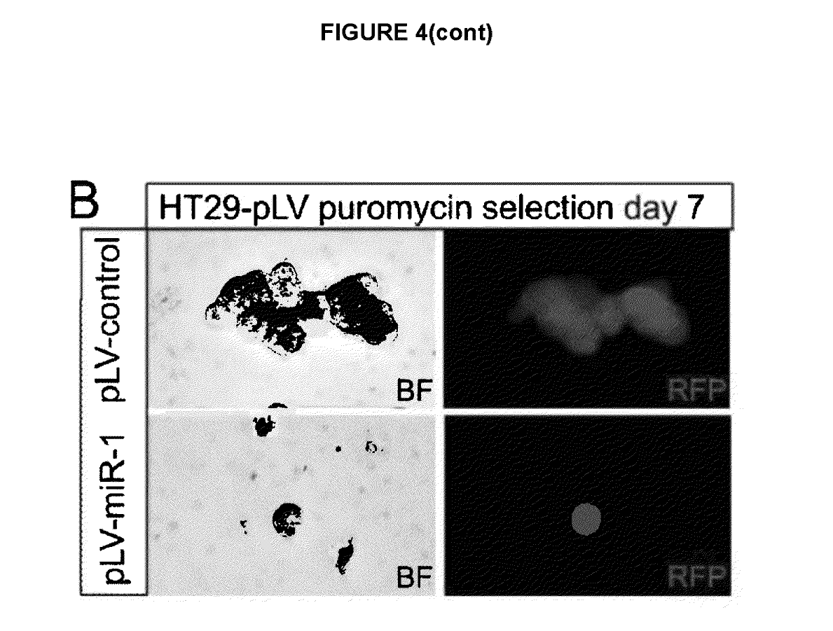

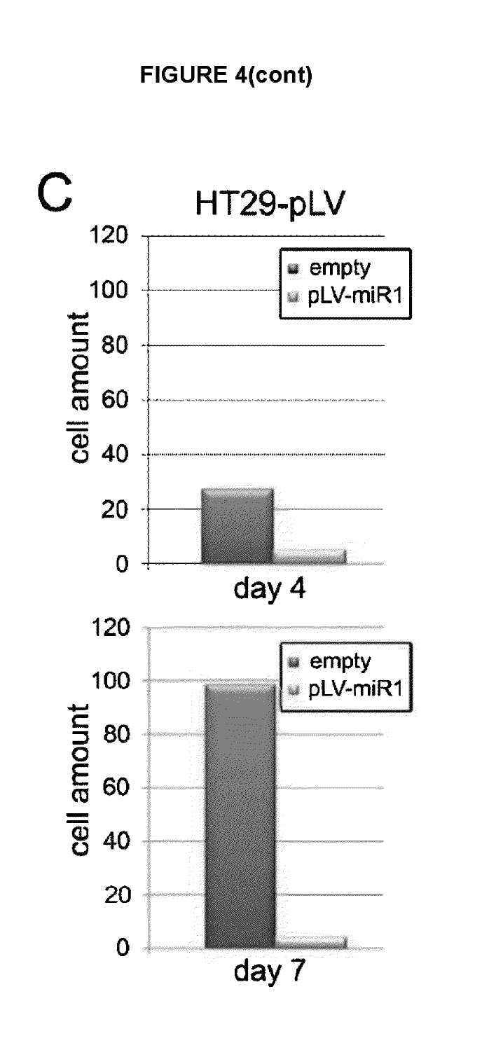

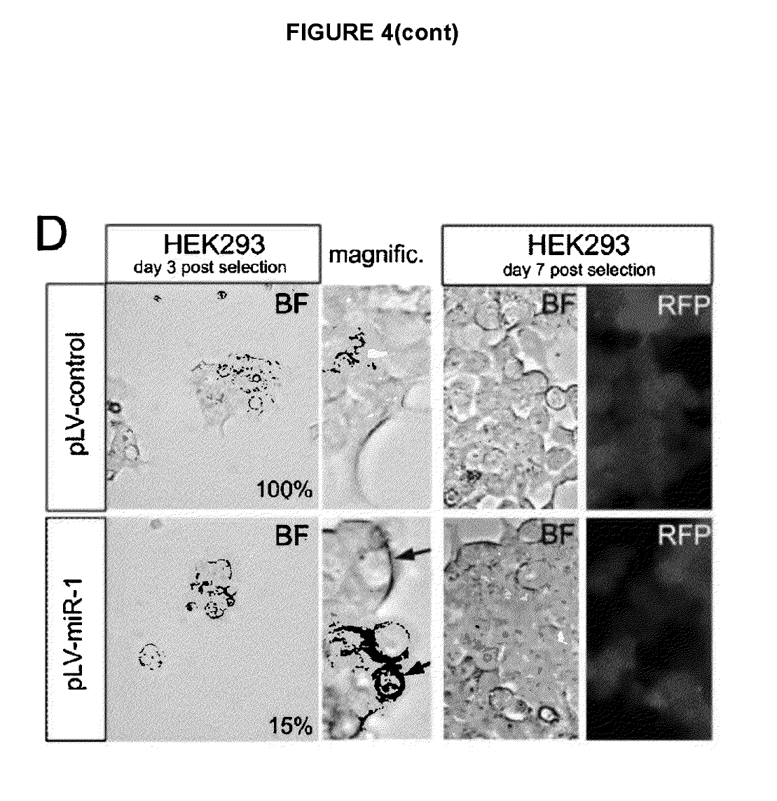

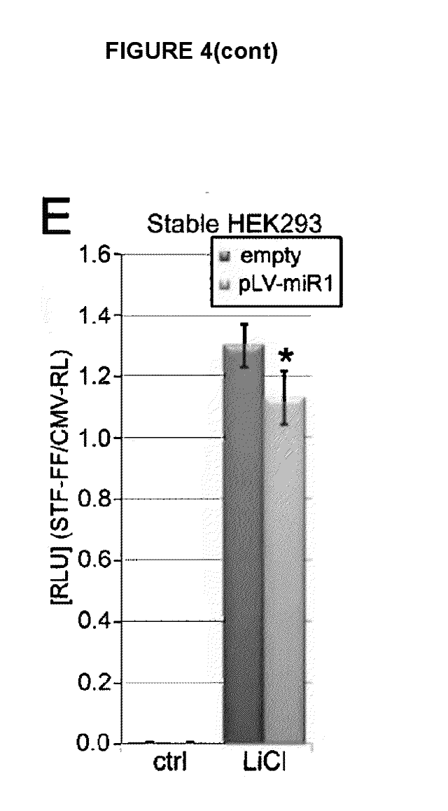

We also investigated the potential function of the candidate Wnt-inhibitor miR-1 in the Wnt-dependent HT29 cancer cell line, because miR-1 was identified as one of the strongest repressors of Wnt-3a-induced activation of the STF reporter (FIG. 4). HT29 cells stably expressing intronic miR-1 in the 5′-UTR of rPURO, a red fluorescent puromycin-N-acetyl-transferase, were generated. These cells express red fluorescence and exhibit puromycin resistance. Hsa-miR-1 expressing cells displayed markedly reduced viability at day 4. While control-virus infected HT29 cells exhibited normal proliferation and colony-formation efficiency at day 7, Pre-miR-1 expressing HT29 cells did not show any obvious signs of proliferation (FIG. 4A-C). HEK293 cells that stably express hsa-miR-1, while showed an initial reduction in cell proliferation at day 4 of selection compared to control, did not display any major proliferation defect by day 7. These results may suggest that the compromised viability in HT29 cells, as compared to HEK293 cells, can be due to a specific Wnt-dependence of HT29 colon cancer cells for their survival (FIG. 4D).

miR-1 Inhibits Expression of a Wnt-Responsive Reporter (Conductin-lacZ) in Primary Mammary Organoids.

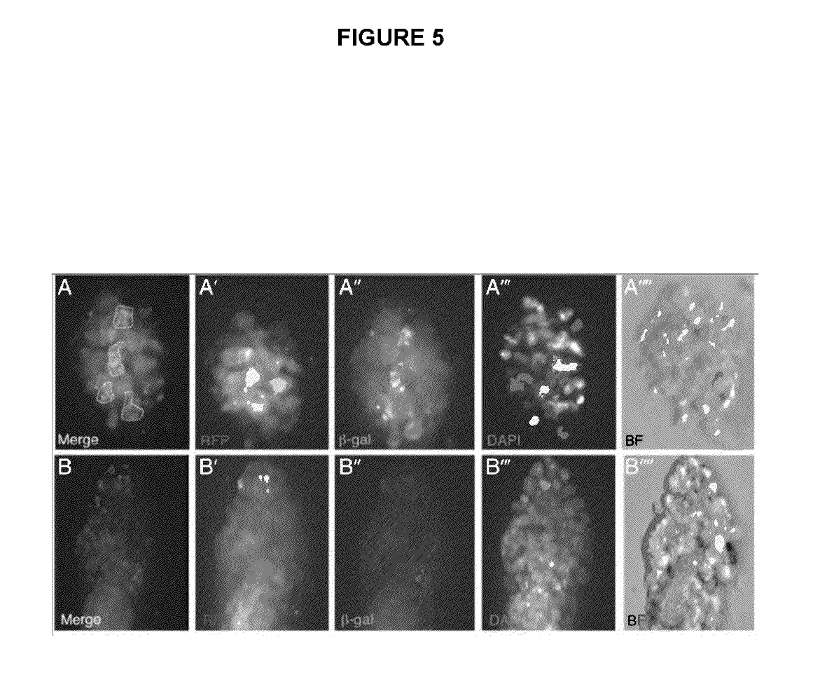

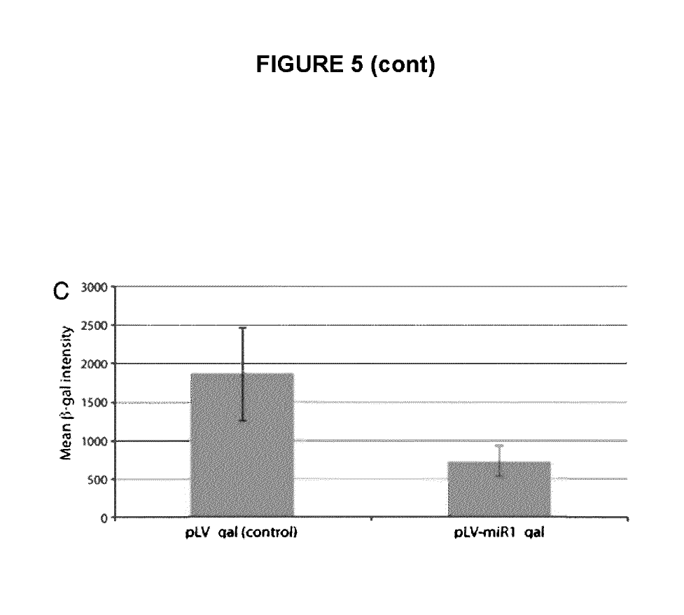

To test whether candidate miRs can influence Wnt signaling in an in vivo context we derived primary mammary epithelial organoids from the axin2/conductin-lacZ mouse [37] using protocols described in Teissedre et al. [38]. Conductin-lacZ has been previously shown to respond to activated-Wnt signaling in mammary epithelial tissue. We introduced a miR-1 expression construct into mammary epithelial organoids derived from the conductin-lacZ in vivo reporter mouse using lentiviral transduction (pLV-miR-1 from Biosettia Inc., USA) and investigated whether expression of miR-1 could influence the expression of the β-gal reporter compared to pLV-empty vector control. As shown in FIG. 5, while pLV control vector (as followed by DsRED expression) did not influence the expression of β-gal reporter (green) (FIG. 5A-A″″), expression of miR-1 within the organoids strongly repressed reporter activity (FIG. 5B-B″″) (see quantification in FIG. 5C). In fact in most cases, miR-1 transduced organoid colonies exhibited almost no immuno-reactivity towards β-gal under identical exposure conditions. These data strongly suggest that ectopic expression of miR-1 may be sufficient in inhibiting the Wnt-responsive reporter in an in vivo context.

### Discussion

In this report we provide the results from a comprehensive screen for the identification, and validation/characterization of human Wnt pathway-modulating miRNAs using a systematic HTS approach. Three candidate Wnt-repressing miRs, namely, miR-1, miR-25 and miR-613, were further characterized in cell-biological assays. In addition to the known Wnt-modulatory miRNAs, such as miR-200a [29], [32], Drosophila miR-315 [28], miR-8 [30], miR-27 [31], zebrafish miR-203 [33], and miR-34 [34], we identified 37 additional miRs that modulate the activity of the Wnt-pathway reporter in cultured human cells. The functions of these 37 miRs, their potential evolutionary conservation, as well as their putative target genes can now be addressed in future studies.

Notably, we uncovered an interesting correlation between many of the candidate miRs that exhibit sequence similarities, both within and outside their mature seed sequence, and their ability to exert similar modulating influence on the activity of the Wnt3a/β-catenin pathway, thereby indicating sequence specificity in miR-mediated Wnt-pathway modulation. The consensus sequences may indicate novel functional seed and “co-seed” sequences that may be involved in the modulation of the Wnt pathway. The partly disrupted consensus seed and co-seed sequences might reflect the imperfect base-pairing with their cognate target genes that participate in Wnt signaling. Moreover, alignment of miRs that could regulate the Wnt reporter made intra- and inter-family related functional consensus sequences apparent (i.e. the seed of miR-1 and miR-613 or within the miR-302 and -515 families (FIG. 13)). This allows the generation of testable hypotheses regarding the functional relevance of specific nucleotide substitutions in miRs within the same family (FIG. 9).

Interestingly, our data also revealed that Wnt-inhibitory miRs tend to be anti-oncomiRs and Wnt-activating miRs tend to be oncomiRs. While the target-directness of each miR needs to be identified and validated in future studies, these data suggest that oncomiRs could contribute to elevated Wnt-pathway activity in cancers (FIG. 1), whereas anti-oncomiRs could function in buffering high levels of Wnt activity by keeping the expression of pathway components in check. Deregulated miR expression profiles might also contribute to oncogenesis by repressing the tumor suppressors, p21/p53 (miR-25 [42], [43], miR-504 [44]) which in turn might affect Wnt signaling. Disruption of cell cycle regulation may also stem from deregulated Wnt activity [45]. As the Wnt pathway is known to be pro-proliferative, and has target genes like c-myc and cyclin-D1 [24], [46], in certain contexts including cancers, the fine-tuning or buffering of Wnt activity by several distinct miRs could modulate proliferative potential of tumor cells, which might also explain the observed correlation. Further studies addressing the identities of the direct targets of candidate miRs can clarify their precise function(s) in the regulation of the Wnt pathway in oncogenesis. This could be addressed in global biochemical approaches such as SILAC or AGO-RIPA (also via HITS/PAR-CLIP [47], [48]) in cytosolic DGCR8 or Drosha loss of function cells transfected with synthetic miRs to identify direct targets.

3 out of 38 candidate miRs (miR-1, miR-25, miR-613) were further characterized in Wnt-responsive cultured cells and all were validated for their Wnt-inhibitory properties identified in the initial screen. Epistasis experiments revealed that candidate miRs target the signaling network at different sites. Pre-mir-1 may function most upstream, followed by miR-613 and then miR-25, which seems to influence the most downstream activity at the level of J3-catenin. All miRs down-regulated Wnt3a-CM and LiCl induced Wnt pathway activity, while only Pre-miR-25 was able to repress Axin1+2-siRNAs or β-catenin-S37A induced activity. Hsa-Pre-mir-1 had a lesser ability to reduce total β-catenin protein levels under conditions of high pathway activation with LiCl. Interestingly, the results from this set of epistasis experiments are in agreement with a known function of Axin downstream/independent of the GSK3 β-catenin destruction complex (FIG. 1C), probably via nuclear-export mediated reduction (FIG. 1B, inset). Hsa-miR-1 and -613 seem to be not closely/directly related but share identical seed sequences and act upstream of Axin and probably downstream of GSK3 (as judged by their inhibitory effect on LiCl mediated activation of the reporter). Therefore both miRs may target a component of the Wnt-pathway upstream of β-catenin via their identical seed sequence, although the precise and relevant molecular targets remain to be identified. That said, it is important to note that cMET has been previously suggested to be a direct target of miR-1 [49], [50], [51].

Analyses of miR-25 function in the regulation of the Wnt pathway suggests a potential function in the translational inhibition of β-catenin via its binding to the β-catenin coding sequence and not its 3′-UTR. Expression of miR-25 repressed the psi-check2 sensor containing the miR-25 binding site, and moderately reduced β-catenin protein levels, while β-catenin transcript levels remained unchanged. Curiously the effect of hsa-miR-25 on β-catenin seems to be more effective under conditions of high pathway and low destruction complex activity, when translational differences preponderate and come into play due to strongly reduced post-translational regulation of β-cat. Reduced β-cat protein amounts can thus be better resolved in LiCl-induced HEK293 cells with high pathway activity, while a low pathway activity by Wnt-3a-CM (ca. 10-fold in STF assay, LiCl ca.50-100-fold) could only be reduced by miR-1 and miR-613 that can block a more upstream part of the pathway and are thus more efficient (FIG. 3D). A downstream role of miR-25 is also in agreement with a Drosophila miR-25/92 evolutionarily related cluster (FIG. 7) that can target the Wnt/Wg pathway (Pancratov and DasGupta, unpublished data). Recent evidence also suggests that miR-25 may inhibit Wnt/β-catenin dependent cancer viability by targeting Pcaf [52] which binds, acetylates, stabilizes and activates β-cat [53], thereby corroborating our observation that the influence of miR-25 is likely at the level of β-cat. Intriguingly, miR-32, that inherits the same seed as miR-25 also targets Pcaf [52] but instead, upregulates the Wnt-reporter in our reporter assays (FIG. 9). These observations suggest that the coding sequence of β-catenin itself may be the primary and direct target of miR-25. Additionally, the opposite effects of miR-25 and miR-32 on the Wnt reporter may imply an important and distinguishing role for the co-seed sequence of miR-25, which is strongly divergent between miR-25 and miR-32 (FIG. 9).

Finally, the very strong anti-proliferative effect of hsa-miR-1 in Wnt/β-catenin dependent human cancer cells (HT29) but not in HEK293 cells, combined with its strong inhibitory effect on an in vivo Wnt-reporter in primary mammary epithelial organoids (FIG. 5), and its lack of known oncogenic properties, highlight its potential as a novel miRNA-based candidate for the development of anti-cancer therapies. Upstream inhibitors like miR-1 besides downstream inhibitors like miR-25 thus show interesting properties for anti-cancer treatments in Wnt-dependent cancers and further support current findings that upstream components of the Wnt pathway are also valid and rational targets for cancer-therapies, even in cells with downstream mutations [15], [54], [55].

In summary our study reports the first comprehensive identification of Wnt-modulating miRs in human cells and represents how miR-based HTS can be employed as a powerful tool to systematically identify pathway relevant miRs. Since pathway-modulatory miRs could have functional impact in cancers associated with deregulated cell signaling, these findings could also benefit the long-term goal of developing miR-based therapeutics and for the diagnostic classification of cancers by expression profile signatures.

### REFERENCES

1. Moser A R, Luongo C, Gould K A, McNeley M K, Shoemaker A R, et al. (1995) ApcMin: a mouse model for intestinal and mammary tumorigenesis. Eur J Cancer 31A, 1061-1064.
2. Bartel D P (2004) MicroRNAs: genomics, biogenesis, mechanism, and function Cell 116, 281-297.
3. Liu Q, Paroo Z (2010) Biochemical Principles of Small RNA Pathways. Annu Rev Biochem.
4. Tay Y, Zhang J, Thomson A M, Lim B, Rigoutsos I (2008) MicroRNAs to Nanog, Oct4 and Sox2 coding regions modulate embryonic stem cell differentiation. Nature 455, 1124-1128.
5. Visone R, Croce C M (2009) MiRNAs and cancer. Am J Pathol 174, 1131-1138.
6. Liu W, Mao S Y, Zhu W Y (2007) Impact of tiny miRNAs on cancers World. J Gastroenterol 13, 497-502.
7. O'Day E, Lal A (2010) MicroRNAs and their target gene networks in breast cancer. Breast Cancer Res 19; 12, 201.
8. Iorio M V, Ferracin M, Liu C G, Veronese A, Spizzo R, et al. (2005) MicroRNA gene expression deregulation in human breast cancer Cancer Res 65, 7065-7070.
9. Adams, B D, Guttilla, I K, White B A (2008) Involvement of microRNAs in breast cancer. Semin Reprod Med 26, 522-536.
10. Baffa R, Fassan M, Volinia S, O'Hara B, Liu C G, et al. (2009) MicroRNA expression profiling of human metastatic cancers identifies cancer gene targets. J Pathol 219, 214-221.
11. Johnson, S M, Grosshans H, Shingara J, Byrom M, Jarvis R, et al. (2005) RAS is regulated by the let-7 microRNA family. Cell 120, 635-647.
12. Le M T, The C, Shyh-Chang N, Xie H, Zhou B, et al. (2009) MicroRNA-125b is a novel negative regulator of p53 Genes Dev 23, 862-876.
13. Zhang Y, Gao J S, Tang X, Tucker L D, Quesenberry P et al., (2009) MicroRNA 125a and its regulation of the p53 tumor suppressor gene. FEBS Lett 19; 583, 3725-3730.
14. Liu C C, Prior J, Piwnica-Worms D, Bu G (2010) LRP6 overexpression defines a class of breast cancer subtype and is a target for therapy Proc Natl Acad Sci USA 107, 5136-5141.
15. He B, Reguart N, You L, Mazieres J, Xu Z, et al. (2004) A monoclonal antibody against Wnt-1 induces apoptosis in human cancer cells Neoplasia 6, 7-14.
16. Zhang J, Li Y, Liu Q, Lu W, Bu G (2010) Wnt signaling activation and mammary gland hyperplasia in MMTV-LRP6 transgenic mice: implication for breast cancer tumorigenesis. Oncogene 29, 539-549.
17. Bjorklund P, Svedlund J, Olsson A K, Akerstrom G, Westin G. (2009) The internally truncated LRP5 receptor presents a therapeutic target in breast cancer. PLoS One 4, e4243.
18. Luu H H, Zhang R, Haydon R C, Rayburn E, Kang Q, et al. (2004) Wnt/beta-catenin signaling pathway as a novel cancer drug target. Curr Cancer Drug Targets 4, 653-671.
19. Quaiser T, Anton R, Kuhl M (2006) Kinases and G proteins join the Wnt receptor complex. Bioessays 28, 339-343.
20. Ryu M J, Cho M, Song J Y, Yun Y S, Choi I W, et al. (2008) Natural derivatives of curcumin attenuate the Wnt/beta-catenin pathway through down-regulation of the transcriptional coactivator p300. Biochem Biophys Res Commun 377, 1304-1308.
21. Wieczorek M, Paczkowska A, Guzenda P, Majorek M, Bednarek A K, et al. (2008) Silencing of Wnt-1 by siRNA induces apoptosis of MCF-7 human breast cancer cells. Cancer Biol Ther 7, 268-274.
22. Hsi L C, Angerman-Stewart J, Eling T E (1999) Introduction of full-length APC modulates cyclooxygenase-2 expression in HT-29 human colorectal carcinoma cells at the translational level. Carcinogenesis 20, 2045-2049.
23. Chen J S, Liang Q M, Li H S, Yang J, Wang, S, et al. (2009) Octreotide inhibits growth of colonic cancer SW480 cells by modulating the Wnt/β-catenin pathway. Pharmazie 64, 126-131. 29.
24. Shan B E, Wang M X, Li R Q (2009) Quercetin inhibit human SW480 colon cancer growth in association with inhibition of cyclin D1 and survivin expression through Wnt/beta-catenin signaling pathway. Cancer Invest 27, 604-612.
25. Lu W, Tinsley H N, Keeton A, Qu Z, Piazza G A, et al. (2009) Suppression of Wnt/beta-catenin signaling inhibits prostate cancer cell proliferation. Eur J Pharmacol 602, 8-14.
26. Krutzfeldt J, Rajewsky N, Braich R, Rajeev K G, Tuschl T et al. (2005) Silencing of microRNAs in vivo with ‘antagomirs’. Nature 438, 685-689.
27. Tong A W, Nemunaitis J (2008) Modulation of miRNA activity in human cancer: a new paradigm for cancer gene therapy? Cancer Gene Ther 15, 341-355.
28. Silver S J, Hagen J W, Okamura K, Perrimon N, Lai E C (2007) Functional screening identifies miR-315 as a potent activator of Wingless signaling Proc Natl Acad Sci USA 104:18151-6.
29. Xia H, Ng S S, Jiang S, Cheung W K, Sze J, et al. (2010) miR-200a-mediated downregulation of ZEB2 and CTNNB1 differentially inhibits nasopharyngeal carcinoma cell growth, migration and invasion. Biochem Biophys Res Commun 391, 535-541.
30. Kennell J A, Gerin I, MacDougald O A, Cadigan K M (2008) The microRNA miR-8 is a conserved negative regulator of Wnt signaling. Proc Natl Acad Sci USA 105, 15417-15422.
31. Wang T, Xu Z (2010) miR-27 promotes osteoblast differentiation by modulating Wnt signaling. Biochem Biophys Res Commun.
32. Saydam O, Shen Y, Würdinger T, Senol O, Broke E, et al. (2009) Downregulated microRNA-200a in meningiomas promotes tumor growth by reducing E-cadherin and activating the Wnt/beta-catenin signaling pathway. Mol Cell Biol 29, 5923-5940.
33. Thatcher E J, Paydar I, Anderson K K, Patton J G (2008) Regulation of zebrafish fin regeneration by microRNAs. Proc Natl Acad Sci USA 10518384-9.
34. Hashimi S T, Fulcher J A, Chang M H, Gov L, Wang S, et al. (2009) MicroRNA profiling identifies miR-34a and miR-21 and their target genes JAG1 and WNT1 in the coordinate regulation of dendritic cell differentiation. Blood 114:404-14.
35. Dasgupta R, Kaykas A, Moon R T, Perrimon N (2005) Functional genomic analysis of the Wnt-wingless signaling pathway. Science 308, 826-833.
36. Willert K, Brown J D, Danenberg E, Duncan A W, Weissman I L, et al. (2003) Wnt proteins are lipid-modified and can act as stem cell growth factors. Nature 423, 448-452.
37. Lustig B, Jerchow B, Sachs M, Weiler S, Pietsch T, et al. (2002) Negative feedback loop of Wnt signaling through upregulation of conductin/axin2 in colorectal and liver tumors. Mol Cell Biol. 22:1184-93.
38. Teissedre B., Pinderhughes A, Incassati A, Hatsell S J, Hiremath M, et al. (2009) MMTV-Wnt1 and -ΔN8913-catenin induce canonical signaling in distinct progenitors and differentially activate Hedgehog signaling within mammary tumors. PLoS One. 4:e4537.
39. Ventura A, Young A G, Winslow M M, Lintault L, Meissner A, et al., (2008) Targeted deletion reveals essential and overlapping functions of the miR-17 through 92 family of miRNA clusters. Cell. 132:875-86
40. Krieghoff E, Behrens J, Mayr B (2006) Nucleo-cytoplasmic distribution of beta-catenin is regulated by retention. J Cell Sci 119, 1453-1463.
41. Cong F, Varmus H (2004) Nuclear-cytoplasmic shuttling of Axin regulates subcellular localization of beta-catenin. Proc Natl Acad Sci USA 101, 2882-2887.
42. Kan T, Sato F, Ito T, Matsumura N, David S, et al. (2009) The miR-106b-25 polycistron, activated by genomic amplification, functions as an oncogene by suppressing p21 and Bim. Gastroenterology 136, 1689-1700.
43. Kumar M, Lu Z Takwi A A, Chen W, Callander N S, et al. (2010) Negative regulation of the tumor suppressor p53 gene by microRNAs. Oncogene.
44. Hu W, Chan C S, Wu R, Zhang C, Sun Y, et al. (2010) Negative regulation of tumor suppressor p53 by microRNA miR-504. Mol Cell 38, 689-699.
45. Davidson G, Shen J, Huang Y L, Su Y, Karaulanov E, et al., (2009) Cell cycle control of wnt receptor activation. Dev Cell 17:788-99.
46. Xu W L, Wang Q, Du M, Zhao Y H, Sun X R, et al. (2010) Growth inhibition effect of β-catenin small interfering RNA-mediated gene silencing on human colon carcinoma HT-29 cells. Cancer Biother Radiopharm 25:529-37.
47. Chi S W, Zang J B, Mele A, Darnell R B (2009) Argonaute HITS-CLIP decodes microRNA-mRNA interaction maps. Nature 460, 479-486.
48. Hafner M, Landthaler M, Burger L, Khorshid M, Hausser J, et al. (2010) Transcriptome-wide identification of RNA-binding protein and microRNA target sites by PAR-CLIP. Cell 141, 129-141.
49. Yan D, Dong Xda E, Chen X, Wang L, Lu et al. (2009) MicroRNA-1/206 targets c-Met and inhibits rhabdomyosarcoma development. J Biol Chem 284:29596-604.
50. Tuynman J B, Vermeulen L, Boon E M, Kemper K, Zwinderman A H, et al. (2008) Cyclooxygenase-2 inhibition inhibits c-Met kinase activity and Wnt activity in colon cancer. Cancer Res. 68:1213-20.
51. Herynk M H, Tsan R, Radinsky R, Gallick G E (2003) Activation of c-Met in colorectal carcinoma cells leads to constitutive association of tyrosine-phosphorylated beta-catenin. Clin Exp Metastasis 20:291-300.
52. Pichiorri F, Suh S S, Ladetto M, Kuehl M, Palumbo T, et al. (2008) MicroRNAs regulate critical genes associated with multiple myeloma pathogenesis. Proc Natl Acad Sci USA 105, 12885-12890.
53. Ge X, Jin Q, Zhang F, Yan T, Zhai Q (2009) PCAF acetylates β-catenin and improves its stability. Mol Biol Cell 20, 419-427.
54. He B, Requart N, You L, Mazieres J, Xu Z, et al. (2005) Blockade of Wnt-1 signaling induces apoptosis in human colorectal cancer cells containing downstream mutations. Oncogene 24, 3054-3058.
55. Kim S Y, Dunn I F, Firestein R, Gupta P, Wardwell L, et al. (2010) CSKlepsilon is required for breast cancers dependent on b-catenin activity. Plos One. 5(2); e8979.

What is claimed is:

1. A method for modulating Wnt signaling pathways in a cell, the method comprising contacting the cell with at least one microRNA listed in FIG. 1 or FIG. 2 or expressing at least one microRNA listed in FIG. 1 or 2 in the cell, wherein the at least one microRNA modulates expression of at least one nucleic acid sequence encoding a component of the Wnt pathway in the cell.

2. The method of claim 1, wherein the cell is a cancer cell.

3. The method of claim 2, wherein the canonical Wnt signaling pathway is elevated or hyper-activated in the cancer cell and the at least one microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38).

4. The method of claim 3, wherein each of the at least one microRNA comprises a consensus sequence as set forth in FIG. 7 or 9.

5. The method of claim 3, wherein each of the at least one microRNA comprises a consensus sequence as set forth in FIG. 7 or 9 and is a member of either of the miR-1/206 family or the miR-25/92 family.

6. The method of claim 5, wherein the consensus sequence is a GGAAUGU seed sequence.

7. The method of claim 6, wherein the at least one microRNA is miR-1 or miR-613.

8. The method of claim 5, wherein the consensus sequence comprises a UUGCAC seed sequence and the microRNA is miR-25, miR-92a, or miR-92b.

9. The method of claim 3, wherein the at least one microRNA is miR-1, miR-25 or miR-613.

10. The method of claim 1, wherein the at least one microRNA is microRNA 196a-1 (SEQ ID NO: 2), 576 (SEQ ID NO: 6), 346 (SEQ ID NO: 8), 504 (SEQ ID NO: 9), 511-2 (SEQ ID NO: 10), 493-3p (SEQ ID NO: 12), 380-5p (SEQ ID NO: 14), 382 (SEQ ID NO: 18), 519e (SEQ ID NO: 21), 519b (SEQ ID NO: 22), 517a (SEQ ID NO: 23), 302a (SEQ ID NO: 24), 302d (SEQ ID NO: 25), 371 (SEQ ID NO: 26), 19b-1 (SEQ ID NO: 28), 555 (SEQ ID NO: 29), 106a (SEQ ID NO: 33), or 512-2-3p (SEQ ID NO: 34).

11. The method of claim 10, wherein the at least one microRNA comprises a consensus sequence as set forth in FIG. 13.

12. The method of claim 11, wherein the at least one microRNA is a member of either of the miR-302 family or the miR-515 family.

13. A method for treating a subject with a cancer associated with an elevated or hyper-activated canonical Wnt signaling pathway, the method comprising administering to the subject at least one inhibitory microRNA shown in either of FIG. 1 or FIG. 2, wherein the at least one inhibitory microRNA reduces or inhibits the elevated or hyper-activated canonical Wnt signaling pathway.

14. The method of claim 13, wherein at least one inhibitory microRNA is microRNA 223 (SEQ ID NO: 1), 139 (SEQ ID NO: 3), 25 (SEQ ID NO: 4), 136 (SEQ ID NO: 5), 375 (SEQ ID NO: 7), 613 (SEQ ID NO: 11), 134 (SEQ ID NO: 13), 23a (SEQ ID NO: 15), 221 (SEQ ID NO: 16), 28 (SEQ ID NO: 17), 617 (SEQ ID NO: 19), 422b (SEQ ID NO: 20), 150 (SEQ ID NO: 27), 126 (SEQ ID NO: 30), 1-2 (SEQ ID NO: 35), 335 (SEQ ID NO: 36), 218-1 (SEQ ID NO: 37), or 9 (SEQ ID NO: 38).

15. The method of claim 13, wherein at least one inhibitory microRNA comprises a consensus sequence as set forth in FIG. 7 or 9 and is a member of either of the miR-1/206 family or the miR-25/92 family.

16. The method of claim 15, wherein the consensus sequence is a GGAAUGU seed sequence.

17. The method of claim 15, wherein the at least one inhibitory microRNA is miR-1 or miR-613.

18. The method of claim 15, wherein the consensus sequence comprises a UUGCAC seed sequence and the microRNA is miR-25, miR-92a, or miR-92b.

19. The method of claim 15, wherein the at least one inhibitory microRNA is miR-1, miR-25 or miR-613.

20. A method for diagnosing a cancer associated with an elevated or hyper-activated Wnt signaling pathway in a subject, the method comprising determining the level of at least one microRNA shown in either of FIG. 1 or FIG. 2, wherein the at least one microRNA activates the Wnt signaling pathway and detection of upregulated levels of the at least one activator microRNA in the subject is diagnostic for the presence of the cancer in the subject and/or wherein the at least one microRNA inhibits the Wnt signaling pathway, and wherein detection of downregulated levels of the at least one inhibitor microRNA in the subject is diagnostic for the presence of the cancer in the subject.
